(19) United States
(12) Patent Application Publication
(10) Pub. No.: US 2025/0176882 A1
(43) Pub. Date: Jun. 5, 2025

(54) INFORMATION PROCESSING METHODS AND INFORMATION PROCESSING SYSTEM

(71) Applicant: Panasonic Holdings Corporation, Osaka (JP)

(72) Inventors: HIROTO YANAGAWA, Osaka (JP); YUMIKO OHNO, Osaka (JP); NAOMI SATOI, Osaka (JP); HITOSHI TOMINAGA, Kyoto (JP); AKIKO TAKIMOTO, Osaka (JP); SATOSHI FUKUDA, Osaka (JP); YUKIKO UCHIDA, Kyoto (JP); MASATAKA NAKAYAMA, Kyoto (JP)

(21) Appl. No.: 19/033,660

(22) Filed: Jan. 22, 2025

Related U.S. Application Data
(63) Continuation of application No. PCT/JP2023/027119, filed on Jul. 25, 2023.

(30) Foreign Application Priority Data
Aug. 4, 2022 (JP) 2022-124733

(51) Int. Cl. A61B 5/16; A61B 5/00; A61B 5/0205; A61B 5/024; A61B 5/352; A61M 21/00; A61M 21/02; G16H 40/63; G16H 50/30

(52) U.S. Cl. CPC A61B 5/165; A61B 5/0205; A61B 5/352; A61B 5/4836; A61M 21/02; G16H 40/63; G16H 50/30; A61B 5/02405; A61M 2021/0044

(57) ABSTRACT

An information processing method includes receiving or calculating a coefficient of variation of R-R interval (CVRR) of a user and low frequency/high frequency (LF/HP) of the user, and causing a processor to perform a determination process for determining, based on the CVRR and the LF/HF, which of a convergent thinking state, a divergent thinking state, or a relaxed state corresponds to a state related to the user's thinking.

### BACKGROUND

### 1. Technical Field

The present disclosure relates to information processing methods and an information processing system.

### 2. Description of the Related Art

In recent years, there has been active research and development regarding the sensing of human states. The states of a person being sensed are related to cognitive functions, such as an emotional state and a thinking state, for example. If a person's thinking state can be presumed by sensing, it is expected to further improve a person's work productivity.

For example, Japanese Unexamined Patent Application Publication No. 2019-191824 discloses a technique to determine the creativity between a speaker and a listener of a meeting using heart rate data (coefficient of variation of R-R interval (CVRR) or low frequency/high frequency (LF/HF)).

Japanese Unexamined Patent Application Publication No. 2020-8278 discloses a technique for estimating the user's concentration level by combining information on the indoor environment where the user is and the heart rate or brain waves, for example, detected from images obtained by capturing the user.

### SUMMARY

One non-limiting and exemplary embodiment provides an information processing method and the like that determine the states related to examinees' thinking with higher accuracy.

In one general aspect, the techniques disclosed here feature an information processing method according to an aspect of the present disclosure, the information processing method including receiving or calculating a coefficient of variation of R-R interval (CVRR) of a user and a low frequency/high frequency (LF/HF) of the user, and causing a processor to perform a determination process for determining, based on the CVRR and the LF/HF, which of a convergent thinking state, a divergent thinking state, or a relaxed state corresponds to a state related to the user's thinking.

This comprehensive or specific aspect may be realized by a system, device, integrated circuit, computer program, or recording medium such as a computer-readable CD-ROM, or any combination of a system, device, integrated circuit, computer program, and recording medium.

With an information processing method according to the present disclosure, the states related to an examinee's thinking can be determined with higher accuracy.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a diagram for describing an electrocardiogram waveform;

FIG. 1B is a diagram for describing a continuous electrocardiogram waveform;

FIG. 1C is a diagram for describing the spectrum of heart rate fluctuations;

FIG. 2A is a diagram for describing the configuration of an information processing system according to an embodiment;

FIG. 2B is a flowchart illustrating a first example of processes performed by the information processing system according to the embodiment;

FIG. 3 is a flowchart illustrating a second example of processes performed by the information processing system according to the embodiment;

FIG. 4 is a flowchart illustrating a third example of processes performed by the information processing system according to the embodiment;

FIG. 5 includes diagrams for describing examples of states determined by the information processing system according to the embodiment;

FIG. 6 is a flowchart illustrating an example of a process regarding the update of threshold information according to the embodiment;

FIG. 7A is a diagram for describing a first example of the presentation of a determination result according to the embodiment;

FIG. 7B is a diagram for describing a second example of the presentation of determination results according to the embodiment;

FIG. 7C is a diagram for describing a third example of the presentation of determination results according to the embodiment;

FIG. 7D is a diagram for describing a fourth example of the presentation of determination results according to the embodiment;

FIG. 7E is a diagram for describing a fifth example of the presentation of determination results according to the embodiment;

FIG. 7F is a diagram for describing a sixth example of the presentation of determination results according to the embodiment;

FIG. 7G is a diagram for describing a seventh example of the presentation of determination results according to the embodiment;

FIG. 8 is a flowchart illustrating an example of a process for controlling lighting in accordance with a determination result according to the embodiment;

FIG. 9 is a diagram for describing a relationship between the illuminance and color temperature of lighting and a state of a user under lighting control according to the embodiment;

FIG. 10 is a diagram illustrating an example of transfer of pieces of biometric information in an information processing device according to the embodiment; and

FIG. 11 is a diagram illustrating an information processing device according to a modification of the information processing device according to the embodiment.

### DETAILED DESCRIPTIONS

### Underlying Knowledge Forming Basis of the Present Disclosure

The inventors of the present application attempted to examine the behavior of the heart rate of a user in a divergent thinking state on the basis of information regarding the heart rate of an examinee performing a task for inducing the examinee to the divergent thinking state. Similarly, the inventors of the present application attempted to examine the behavior of the heart rate of a user in a convergent thinking state on the basis of information regarding the heart rate of an examinee performing a task for inducing the examinee to the convergent thinking state.

In one experiment, an examinee engaged in six tasks. Two of the six tasks were designed to induce the examinee to the convergent thinking state, and the remaining four tasks were designed to induce the examinee to the divergent thinking state. To minimize the effects of task-induced fatigue, the examinee was asked to take a one-minute break between each task. The order in which the six tasks were performed was designed to be counterbalanced to reduce the impact of the order effect. Note that before engaging in each of the six tasks, the examinee was given an explanation of the task and performed a practice task corresponding to the task for about one minute.

To engage in each task, the examinee followed a procedure of uttering meaningless words for one minute, performing the task for about three minutes, and then uttering meaningless words for one minute. The examinee followed the procedure described above for each of the six tasks. The meaningless words were uttered by the examinee to put the examinee in a neutral thinking state. For each of the six tasks, to ensure that the examinee did not realize that the task was designed to induce the examinee to the convergent thinking state or the divergent thinking state, the names Task A, Task B, Task C, and Task D were used to explain the tasks to the examinee.

One of the two tasks inducing the examinee to the convergent thinking state was a verbal task, and the Japanese version of the Remote Association Task (hereinafter referred to as RAT) was used. The other one was a non-verbal task, and Raven Task (hereinafter referred to as Raven) was used.

The RAT may be a task in which the examinee is presented with three words that may seem to have no commonality at first glance and asked to find a common word associated with each word. For example, the task may be to present the examinee with three words, pure, blue, and fall, and ask the examinee to find a common word associated with the three words, such as water. In this experiment, the RAT was used with reference to the work by R. Orita, M. Hattori, and Y. Nishida titled “Development of a Japanese Remote Associates Task as insight problems”, Japanese J. P sychol. 89, 376-386, (2018).

Raven may be, for example, a task in which the examinee is presented with a silhouette diagram and the user is asked to select the figure that fits the silhouette from candidate figures. In this experiment, Raven was used with reference to the work by L. E. Matzen, Z. O. Benz, K. R. Dixon, J. Posey, J. K. K roger, and A. E. Speed, titled “Recreating Raven's: Software for systematically generating large numbers of Raven-like matrix problems with nor med properties”, Behav. Res. Methods 42, 525-541, 2010.

Two of the four tasks to induce the examinee to the divergent thinking state were verbal tasks, and an Unusual Uses Test (also referred to as UUT) was used. The remaining two of the four tasks were non-verbal tasks, and a Figural Divergent Task (also referred to as FDT) was used.

The UUT may be, for example, a task in which an examinee is asked to name unique ways to use an object. In this experiment, the task in which the examinee was asked to respond with as many unusual uses of a ping pong ball as possible within three minutes was used as the UUT.

The FDT may be, for example, a task in which an examinee is presented with a silhouette diagram and asked to respond with as many interpretations of the silhouette diagram as possible within three minutes.

In this experiment, the task in which the examinee was asked to respond to a figure “+” (in other words, a figure formed by a horizontal line segment and a line segment orthogonal to the line segment) with as many interpretations of the figure as possible within three minutes was used as the FDT.

These experimental results indicate that CVRR tends to be lower when people are in the convergent thinking state, and CVRR tends to be higher when people are in the divergent thinking state or the relaxed state.

Moreover, these experimental results indicate that LF/HF tends to be higher when people are in the divergent thinking state, and LF/HF tends to be lower when people are in the relaxed state.

On the basis of the above-mentioned description, by combining the known fact that it is possible to determine an examinee's degree of relaxation from the values of CVRR and LF/HF (see Japanese Unexamined Patent Application Publication No. 2021-108021) and the above-mentioned experimental results, it may be possible to determine that an examinee is in the convergent thinking state when CVRR is lower than a certain value and in the divergent thinking state or relaxed state when CVRR is higher than the certain value. LF/HF can be used to determine whether the examinee is in the divergent thinking state or the relaxed state, and it may be possible to determine that the examinee is in the relaxed state when LF/HF is lower than a certain value and in the divergent thinking state when LF/HF is higher than the certain value.

Thus, the information processing system according to the present embodiment determines the state related to an examinee's thinking, for example, and more specifically whether an examinee is in the convergent thinking state, the divergent thinking state, or the relaxed state, as follows.
(1) Determine whether or not an examinee is in the convergent thinking state using CVRR.
(2) Determine whether or not the examinee is in the divergent thinking state using CVRR and LF/HF.
(3) Determine whether or not the examinee is one of the convergent thinking state, the divergent thinking state, or the relaxed state using at least N pieces of biometric information out of heart rate (HR), pulse rate, LF/HF, HF, skin temperature, or electrodermal activity. Note that N is an integer greater than or equal to 1, and N is 2, for example.

In the following, biometric information will be described. Biometric information is information corresponding to a person's physiological indicators. Examples of biometric information include electrocardiogram waveform, heart rate, pulse waveform, pulse rate, heart rate interval (R-R interval (RRI)), CVRR, LF/HF, skin temperature, and electrodermal activity. That is, pieces of biometric information may include electrocardiogram waveform, heart rate, pulse waveform, pulse rate, heart rate interval (R-R interval (RRI)), CVRR, LF/HF, skin temperature, and electrodermal activity.

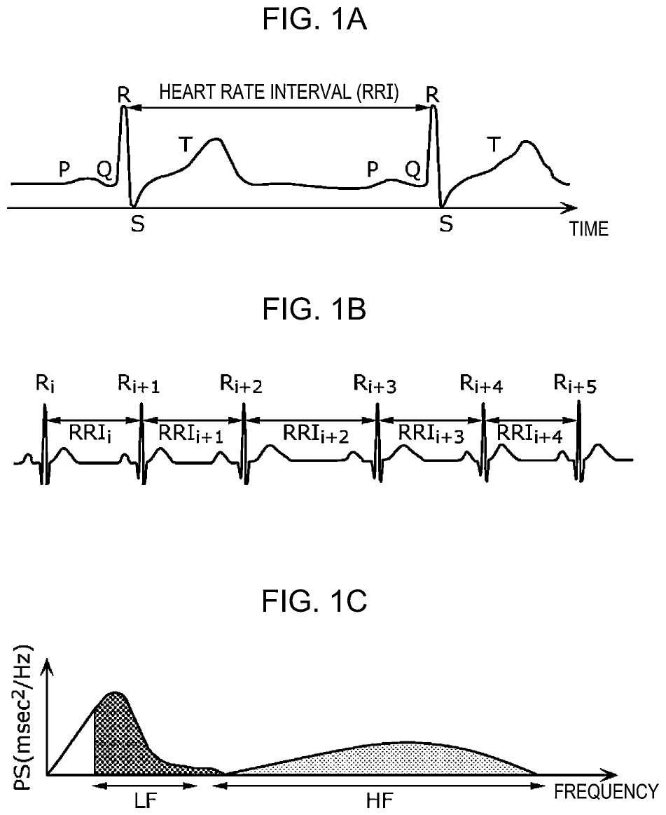

FIG. 1A is a diagram for describing an electrocardiogram waveform. Note that the horizontal axis of FIG. 1A represents time, and the vertical axis thereof represents electric potential difference. An electrocardiogram waveform is also called an electrocardiogram. The electrocardiogram waveform is formed by P wave, Q wave, R wave, S wave, and T wave. The P wave reflects the electrical excitation of the atria. The Q, R, and S waves reflect the electrical excitation of the ventricles. The T wave reflects the process of repolarization of the excited ventricular cardiac myocytes. Among these waves, the R wave has the largest wave height (namely, potential difference) and is the most robust against noise such as myopotential.

The heart rate interval (R-R interval (RRI)) is the time interval between two consecutive R waves in the electrocardiogram waveform. Note that the heart rate is, for example, the number of beats per minute. That is, (heart rate)=60/(the heart rate interval). Note that known methods, such as the Pan and Tompkins method, may be used to detect R waves.

FIG. 1B is a diagram for describing an electrocardiogram waveform. FIG. 1B is a graph illustrating an electrocardiogram waveform. The horizontal axis of the graph represents time, and the vertical axis thereof represents electric potential difference.

As illustrated in FIG. 1B, the electrocardiogram waveform includes a series of R waves, which are Rᵢ, Rᵢ₊₁, Rᵢ₊₂, . . . , and Rᵢ₊₅. The time interval between the peaks of the adjacent R waves Rᵢ and Rᵢ₊ᵢ is RRIᵢ, the time interval between the peaks of the adjacent R waves Rᵢ₊₁ and Rᵢ₊₂ is RRIᵢ₊₁, the time interval between the peaks of the adjacent R waves Rᵢ₊₂ and Rᵢ₊₃ is RRIᵢ₊₂, . . . , the time interval between the peaks of the adjacent R waves Rᵢ₊₄ and Rᵢ₊₅ is RRIᵢ₊₄. Each of these time intervals RRIᵢ, RRIᵢ₊₁, RRIᵢ₊₂, . . . , RRIᵢ₊₄ is a heart rate interval. These heart rate intervals RRIᵢ, RRIᵢ₊₁, RRIᵢ₊₂, . . . , RRIᵢ₊₄ are not equal. That is, these heart rate intervals vary. These variations in heart rate interval are also referred to as heart rate variability or heart rate fluctuations.

CVRR is the coefficient of variation of heart rate fluctuations. CVRR is calculated by dividing the standard deviation of the heart rate intervals by the mean of the heart rate intervals (see (Equation 1)). The standard deviation of the heart rate intervals and the mean of the heart rate intervals may be obtained on the basis of heart rate intervals included in a predetermined period in the electrocardiogram waveform. CVRR may be calculated by performing a time-domain analysis process, for example, on the components of reflected light generated due to irradiation light with which the examinee has been irradiated.

CVRR=(the standard deviation of heart rate intervals)/(the mean of heart rate intervals) (Equation 1)

The RRI is known to reflect the function of the autonomic nervous system. Specifically, it is known that the variation in the R-R interval is small when the examinee is stressed, while the variation in the R-R interval is large when the examinee is relaxed. Thus, the magnitude of CVRR corresponds to the degree of relaxation; in other words, a higher CVRR indicates a higher degree of relaxation.

HR is the number of beats of the examinee per unit time (for example, one minute).

FIG. 1C is a graph illustrating the spectrum of heart rate fluctuations. The horizontal axis of the graph represents frequency, and the vertical axis thereof represents power.

HF and LF are calculated from the power spectral density obtained by performing frequency analysis on equally spaced time series data of heart rate fluctuations using the fast Fourier transform (FFT). Note that a power spectrum may be used instead of the power spectral density. HF is the integral value of the power spectral density in a high frequency range (for example, 0.14 Hz to 0.4 Hz), and it is believed that HF reflects the activity level of the parasympathetic nervous system. LF is the integral value of the power spectral density in a low frequency range (for example, 0.04 Hz to 0.14 Hz), and it is believed that LF reflects the activity level of the sympathetic nervous system and that of the parasympathetic nervous system. Note that the frequency conversion using the FFT may be performed at 5-second intervals or at any time intervals.

LF/HF is said to represent the overall balance between the sympathetic and parasympathetic nervous systems (Japanese Unexamined Patent Application Publication No. 2021-108021). The LF/HF may be equal to (the integral value of the above-mentioned power spectral density at 0.04 Hz to 0.14 Hz)/(the integral value of the above-mentioned power spectral density at 0.14 Hz to 0.4 Hz). Specifically, when the LF/HF, the stress index, is high, the examinee is in a sympathetic dominant state. When the LF/HF, the stress index, is low, the examinee's parasympathetic nervous system is dominant, and the examinee is in a relaxed state.

During the determinations of (1) and (2) above, the examinee wears a sensor capable of measuring heart rate or pulse wave. The above sensor acquires the examinee's biometric information (for example, electrocardiogram waveform), and the acquired electrocardiogram waveform is analyzed using a waveform analysis program to calculate the heart rate or pulse wave. Note that the sensor does not have to be attached to the examinee and may perform non-contact measurements. HF and LF may be calculated by performing a frequency analysis process, for example, on the components of the reflected light generated due to irradiation light with which the examinee has been irradiated. The information processing system determines, at any timing or at any time intervals, which of the convergent thinking state, the divergent thinking state, or the relaxed state the examinee is in. The results of the determinations may be or do not have to be displayed to the examinee. To perform the determinations, a binary determination using a threshold may be performed.

When determining (3) described above, in a case where the examinee is more relaxed in the task execution state than in the resting state, it is determined whether or not the examinee is one of the convergent thinking state, the divergent thinking state, or the relaxed state. The result of the determinations may be or does not have to be displayed to the examinee. Note that in a case where the examinee is determined to be more relaxed in the task execution state than in the resting state, when it is determined that the examinee is one of the convergent thinking state, the divergent thinking state, or the relaxed state, the determinations of (1) and (2) described above may be performed.

In the following, examples of techniques that can be obtained from the content disclosed in the present specification are illustrated, and the effects, for example, obtained from such techniques are described.

(1) An information processing method including receiving or calculating CVRR of a user and LF/HF of the user, and causing a processor to perform a determination process for determining, based on the CVRR and the LF/HF, which of a convergent thinking state, a divergent thinking state, or a relaxed state corresponds to a state related to the user's thinking.

According to the above-mentioned aspect, the state related to the user's thinking can be determined on the basis of the CVRR and LF/HF acquired from the user who is the examinee. Hitherto, there is no known technology for determining the state related to the user's thinking from information regarding a heart rate of the user. According to the above-mentioned information processing method, the state related to the user's thinking can be determined with higher accuracy on the basis of the CVRR and LF/HF that are information regarding the user's heart rate. In this manner, according to the above-mentioned information processing method, the state related to the examinee's thinking, for example, can be determined with higher accuracy.

(2) The information processing method described in (1), in which in the determination process, in a case where the CVRR is less than a first threshold, it is determined that the user is in the convergent thinking state.

According to the above-mentioned aspect, whether or not the user is in the convergent thinking state can be easily determined by using the magnitude determination between the CVRR of the user and the first threshold. In this manner, according to the above-mentioned information processing method, the state related to the examinee's thinking, for example, can be determined more easily with higher accuracy.

(3) The information processing method described in (1) or (2), in which in the determination process, in a case where the CVRR is greater than or equal to a first threshold and where the LF/HF is greater than or equal to a second threshold, it is determined that the user is in the divergent thinking state.

According to the above-mentioned aspect, whether or not the user is in the divergent thinking state can be easily determined by using the magnitude determination between the CVRR of the user and the first threshold and the magnitude determination between the LF/HF of the user and the second threshold. In this manner, according to the above-mentioned information processing method, the state related to the examinee's thinking, for example, can be determined more easily with higher accuracy.

(4) The information processing method described in any one of (1) to (3), in which in the determination process, in a case where the CVRR is greater than or equal to a first threshold and where the LF/HF is less than a second threshold, it is determined that the user is in the relaxed state.

According to the above-mentioned aspect, whether or not the user is in the relaxed state can be easily determined by using the magnitude determination between the CVRR of the user and the first threshold and the magnitude determination between the LF/HF of the user and the second threshold. In this manner, according to the above-mentioned information processing method, the state related to the examinee's thinking, for example, can be determined more easily with higher accuracy.

(5) The information processing method described in any one of (1) to (4), further including, before the determination process, causing the processor to perform a pre-determination process for determining, based on at least the LF/HF, whether or not a state of the user is one of the convergent thinking state, the divergent thinking state, or the relaxed state, and in which in a case where the state of the user is determined to be one of the convergent thinking state, the divergent thinking state, or the relaxed state in the pre-determination process, the determination process is performed.

According to the above-mentioned aspect, on the basis of the CVRR and LF/HF acquired from the user who is the examinee, when it is determined in the pre-determination process that the state related to the user's thinking is one of the convergent thinking state, the divergent thinking state, or the relaxed state, it is then determined which of the three states described above corresponds to the state related to the user's thinking. Thus, the determination result of the state related to the user's thinking can be made more appropriate. If it is to be determined, without performing the pre-determination process, which of the three states described above corresponds to the state related to the user's thinking, an inappropriate determination result may be output in a case where the state related to the user's thinking is none of the three states described above, for example. According to the above-mentioned aspect, after it is determined in the pre-determination process that the state related to the user's thinking is one of the three states described above, it is determined which of the three states described above corresponds to the state related to the user's thinking. Thus, it may be possible to suppress the output of inappropriate determination results, as described above. In this manner, according to the above-mentioned information processing method, the state related to the examinee's thinking, for example, can be determined more appropriately with higher accuracy.

(6) The information processing method described in any one of (1) to (4), further including, before the determination process, causing the processor to perform a pre-determination process for determining, based on at least one of a heart rate, a pulse rate, the LF/HF, HF, a skin temperature, or an electrodermal activity of the user, whether or not the state of the user is one of the convergent thinking state, the divergent thinking state, or the relaxed state, and in which in a case where the state of the user is determined to be one of the convergent thinking state, the divergent thinking state, or the relaxed state in the pre-determination process, the determination process is performed.

According to the above-mentioned aspect, on the basis of the biometric information (namely, at least one of the heart rate, the pulse rate, the LF/HF, the HF, the skin temperature, or the electrodermal activity) acquired from the user who is the examinee, when it is determined in the pre-determination process that the state related to the user's thinking is one of the convergent thinking state, the divergent thinking state, or the relaxed state, it is then determined which of the three states described above corresponds to the state related to the user's thinking. Thus, the determination result of the state related to the user's thinking can be made more appropriate. If it is to be determined, without performing the pre-determination process, which of the three states described above corresponds to the state related to the user's thinking, an inappropriate determination result may be output in a case where the state related to the user's thinking is none of the three states described above, for example. According to the above-mentioned aspect, after it is determined in the pre-determination process that the state related to the user's thinking is one of the three states described above, it is determined which of the three states described above corresponds to the state related to the user's thinking. Thus, it may be possible to suppress the output of inappropriate determination results, as described above. Thus, according to the above-mentioned information processing method, the state related to the examinee's thinking, for example, can be determined more appropriately with higher accuracy.

(7) The information processing method described in (1), in which the CVRR and the LF/HF correspond to a first period included in periods, and in the determination process, based on the CVRR and LF/HF corresponding to each of the periods, the determination process regarding the state related to the user's thinking in the period is performed, the information processing method further including generating a graph representing a percentage of a period determined to be in the convergent thinking state, a percentage of a period determined to be in the divergent thinking state, and a percentage of a period determined to be in the relaxed state among the periods in the determination process, and displaying the graph.

According to the above-mentioned aspect, since a graph representing the percentage of the state related to the user's thinking in each of the periods is displayed, it contributes to allowing the viewer of the graph to grasp the states related to the user's thinking over the periods at a glance. Thus, according to the above-mentioned information processing method, the states related to the examinee's thinking, for example, over the periods can be determined and displayed with higher accuracy, and furthermore this enables the viewer to grasp the states easily.

(8) The information processing method described in (2), further including acquiring identification information for identifying the user, and acquiring threshold information corresponding to the identification information, in which the first threshold is determined based on the threshold information.

According to the above-mentioned aspect, since the first threshold used to perform a magnitude determination on the CVRR of the user is determined on the basis of the threshold information, the first threshold can be made more appropriate, and it can be appropriately determined whether or not the state related to the user's thinking is the convergent thinking state. Thus, according to the above-mentioned information processing method, the state related to the examinee's thinking, for example, can be determined more appropriately with higher accuracy.

(9) The information processing method described in (8), further including acquiring evaluation information obtained by evaluating the state related to the user's thinking using a method different from the determination process, and updating the threshold information based on the determined state of the user and the state of the user indicated by the evaluation information.

According to the above-mentioned aspect, since the first threshold is updated on the basis of the evaluation information obtained by evaluating the state related to the user's thinking by using the method different from the determination process included in the information processing method, this contributes to bringing the determination result of the subsequent state related to the user's thinking closer to the evaluation result obtained using the above-mentioned different method. Thus, according to the above-mentioned information processing method, the state related to the examinee's thinking, for example, can be determined with higher accuracy while bringing the determination result closer to the evaluation result obtained using the above-mentioned different method.

(10) The information processing method described in (3) or (4), further including acquiring identification information for identifying the user, and acquiring threshold information corresponding to the identification information, in which the first threshold and the second threshold are determined based on the threshold information.

According to the above-mentioned aspect, since the first threshold used to perform a magnitude determination on the CVRR of the user and the second threshold used to perform a magnitude determination on the LF/HF of the user are determined on the basis of the threshold information, the first threshold and second threshold can be made more appropriate, and it can be appropriately determined whether or not the state related to the user's thinking is the convergent thinking state or whether or not the state related to the user's thinking is the relaxed state. Thus, according to the above-mentioned information processing method, the state related to the examinee's thinking, for example, can be determined more appropriately with higher accuracy.

(11) The information processing method described in (10), further including acquiring evaluation information obtained by the user evaluating the state related to the user's thinking, and updating the threshold information based on the determined state of the user and the state of the user indicated by the evaluation information.

According to the above-mentioned aspect, since the first threshold is updated on the basis of the subjective evaluation information obtained by the user themselves evaluating the state related to their thinking, this contributes to bringing the determination result of the subsequent state related to the user's thinking closer to the user's evaluation. Thus, according to the above-mentioned information processing method, the state related to the examinee's thinking, for example, can be determined with higher accuracy while bringing the determination result closer to the examinee's subjective evaluation.

(12) The information processing method described in (2), further including, after performing the determination process, performing (i) control to lower a color temperature of illuminating light and increase illuminance of the illuminating light, or (ii) control to lower a color temperature of illuminating light and lower illuminance of the illuminating light on a lighting device illuminating a surrounding area of the user.

According to the above-mentioned aspect, in a case where the state related to the user's thinking is determined to be the convergent thinking state, the user can be appropriately induced to the divergent thinking state or the relaxed state by using the lighting device. Thus, according to the above-mentioned information processing method, the state related to the examinee's thinking, for example, can be determined with higher accuracy, and the user determined to be in the convergent thinking state can be induced to the divergent thinking state or the relaxed state.

(13) The information processing method described in (3), further including, after performing the determination process, performing (i) control to increase a color temperature of illuminating light and increase illuminance of the illuminating light, or (ii) control to lower a color temperature of illuminating light and lower illuminance of the illuminating light on a lighting device illuminating a surrounding area of the user.

According to the above-mentioned aspect, in a case where the state related to the user's thinking is determined to be the divergent thinking state, the user can be appropriately induced to the convergent thinking state or the relaxed state by using the lighting device. Thus, according to the above-mentioned information processing method, the state related to the examinee's thinking, for example, can be determined with higher accuracy, and the user determined to be in the divergent thinking state can be induced to the convergent thinking state or the relaxed state.

(14) The information processing method described in (4), further including, after performing the determination process, performing (i) control to lower a color temperature of illuminating light and increase illuminance of the illuminating light, or (ii) control to increase a color temperature of illuminating light and increase illuminance of the illuminating light on a lighting device illuminating a surrounding area of the user.

According to the above-mentioned aspect, in a case where the state related to the user's thinking is determined to be the relaxed state, the user can be appropriately induced to the convergent thinking state or the divergent thinking state by using the lighting device. Thus, according to the above-mentioned information processing method, the state related to the examinee's thinking, for example, can be determined with higher accuracy, and the user determined to be in the relaxed state can be induced to the convergent thinking state or the divergent thinking state.

(15) An information processing method including, by using a processor, acquiring target information indicating a target state that is a state targeted by a user, acquiring biometric information of the user, determining a state related to the user's thinking based on the biometric information, comparing the target state indicated by the target information with the determined state related to the user's thinking, and in a case where, as a result of the comparison, the target state and the determined state related to the user's thinking are different from each other, performing control to change characteristics of illuminating light emitted from a lighting device illuminating a surrounding area of the user.

According to the above-mentioned aspect, in a case where the state related to the user's thinking determined from the biometric information of the user is different from the target state, the state related to the user's thinking can be appropriately guided to the target state by using the lighting device. Thus, according to the above-mentioned information processing method, the examinee's creative thinking state can be objectively evaluated, and the state related to the user's thinking can be guided to the target state.

(16) An information processing system including a light source that irradiates a user with irradiation light, a detector that detects reflected light generated due to the irradiation light, a processing circuit, a memory device, and a display device, in which the processing circuit calculates a CVRR corresponding to heart rate fluctuations or pulse fluctuations of the user by performing a time-domain analysis process on a component of the reflected light detected by the detector over a unit period of time, calculates an LF/HF corresponding to the heart rate fluctuations or pulse fluctuations of the user by performing a frequency analysis process on the component of the reflected light detected by the detector over the unit period of time, acquires threshold information indicating a first threshold and a second threshold from the memory device, performs a first determination process for determining whether or not a mean value of the CVRR in the unit period of time is greater than or equal to the first threshold, performs a second determination process for determining whether or not a mean value of the LF/HF in the unit period of time is greater than or equal to the second threshold, performs a third determination process for determining, based on the first determination process and the second determination process, which of a divergent thinking state, a convergent thinking state, or a relaxed state the user is in during the unit period of time, generates an image related to the state of the user determined in the third determination process, and causes the display device to display the generated image.

According to the above-mentioned aspect, substantially the same effects as the above-mentioned information processing methods are achieved.

The embodiments described below are all examples, each of which is either comprehensive or specific. The numerical values, shapes, materials, structural elements, structural-element arrangement positions and connection forms, steps, and sequences of steps illustrated in the following embodiments are examples and are not intended to limit the technology of the present disclosure. Among the structural elements in the following embodiments, the structural elements that are not described in the independent claims that indicate the highest level of concept are described as optional structural elements. Each diagram is schematic and not necessarily depicted in a strictly detailed manner. Furthermore, in each diagram, substantially identical or similar structural elements are denoted by the same signs. Redundant description may be omitted or simplified.

In the present disclosure, all or some of the circuits, units, devices, members, or all or some of portions, or all or some of functional blocks in the block diagrams can be implemented by one or more electronic circuits including, for example, a semiconductor device, a semiconductor integrated circuit (IC), or a large scale integration (LSI). The LSI or IC may be integrated into a single chip or may be formed by a combination of chips. For example, functional blocks other than memory elements may be integrated on a single chip. In this case, the term such as LSI or IC is used; however, the term varies depending on the degree of integration and may be called system LSI, very large scale integration (VLSI), or ultra large scale integration (ULSI). Field programmable gate arrays (FPGAs), which are programmed after the LSI is manufactured, or reconfigurable logic devices, which can reconfigure the junction relationships inside the LSI or set up circuit partitions inside the LSI, can be used for the same purpose.

In the embodiments described below, it is assumed that examinees are office workers, learners in educational facilities or learning environments, and so forth. The assumed office workers do not perform physical tasks that require work results from physical movements, but perform desk work tasks that require intellectual productivity, such as document preparation, information management, classification work, new idea generation, and new project proposals. The tasks performed by the assumed office workers include intellectual productivity tasks performed by individuals and may also include intellectual productivity tasks performed by a group of individuals, a typical example of which is brainstorming. The term “desk work task” is not necessarily limited to seated task, but can include tasks such as standing creative meetings, and can also include tasks performed by a group of individuals.

Intellectual productivity is affected not only by the knowledge or skill capacity of an individual or a group of individuals, but also by their thinking state. Specifically, intellectual productivity requires both convergent thinking and divergent thinking.

Convergent thinking is logical thinking or reasoning from known information to arrive at a solution. Specifically, thinking about problems with correct answers, such as crossword puzzles and calculation problems, falls under convergent thinking.

Additionally, the process of summarizing ideas generated through brainstorming to reach one or more conclusions falls under convergent thinking. Convergent thinking can also be referred to as critical thinking.

In contrast, divergent thinking is the process of generating new ideas by thinking in various ways from known information. Specifically, the way of thinking to create new ideas for which there is no right answer falls under divergent thinking. Additionally, the way of thinking used to create ideas, such as brainstorming, falls under divergent thinking. Divergent thinking can also be referred to as creative thinking.

The inventors of the present application have come up with a method in which a task requiring convergent thinking and a task requiring divergent thinking are assigned to an examinee and it can be determined which of a convergent thinking state, a divergent thinking state, or a relaxed state a person is in on the basis of the biometric information before, during, and after the intellectual tasks.

In the present specification, the convergent thinking state refers to a state in which a person is engaged in convergent thinking. In this case, the convergent thinking state may include a state in which a person is focused. The divergent thinking state refers to a state in which a person is engaged in divergent thinking. In this case, the divergent thinking state may include a state in which a person is being creative.

Examinees are not limited to office workers, but can also be learners at school or at home.

The information processing device according to the present embodiment measures biometric information during intellectual productivity tasks in order to determine convergent thinking and divergent thinking during the intellectual productivity tasks. The biometric information includes various indicators such as heart rate, electrocardiogram waveform, pulse wave, skin potential, electrodermal activity, and skin temperature. The biometric information is physiological data that can be measured during the work of intellectual productivity workers. On the basis of feature values obtained from biometric information including CVRR and LF/HF among these pieces of biometric information, the above-mentioned information processing device calculates evaluation information regarding the convergent thinking state, divergent thinking state, or relaxed state.

Intellectual productivity tasks are also called tasks, and the tasks may be examinees' usual tasks or cognitive tasks used in psychological experiments, for example. The information processing system calculates, on the basis of each examinee's biometric information, evaluation information regarding whether the examinee is in the convergent thinking state, the divergent thinking state, or the relaxed state.

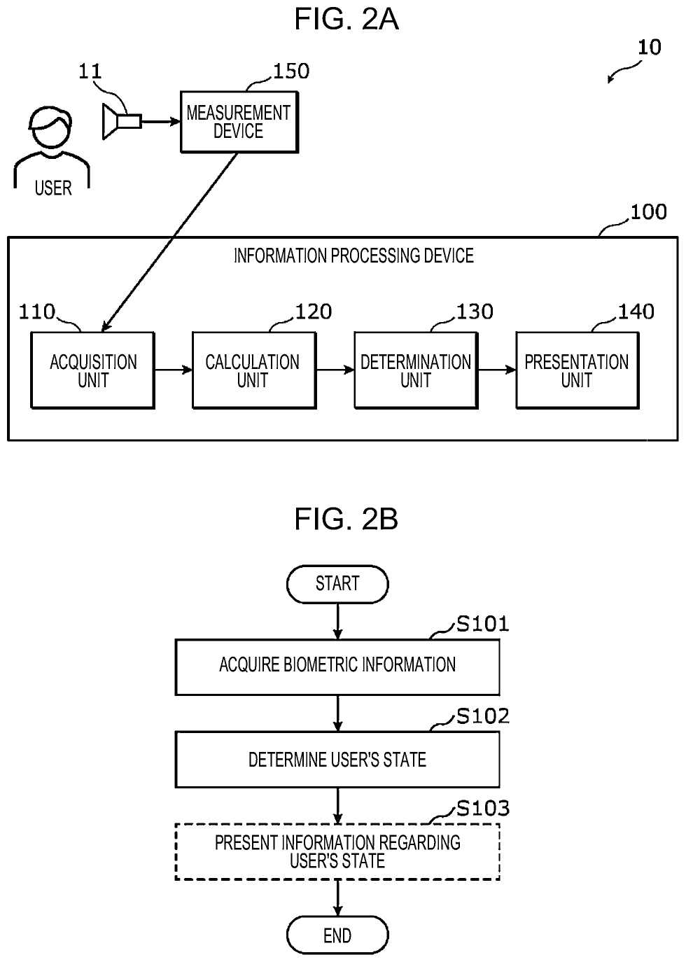

FIG. 2A is a diagram for describing the configuration of an information processing system 10 according to the present embodiment, and illustrates the configuration of conceptual functional units of the information processing system 10.

Note that the functional units generally refer to logically separable software programs (computer programs) or components such as hardware devices. Thus, the functional units according to the present embodiment refer not only to modules in computer programs but also to modules in hardware configurations. Thus, the present embodiment includes computer programs for causing these functional units to function (programs for causing a computer to perform the respective procedures, programs for causing a computer to function as the respective means, programs for causing a computer to realize the respective functions), as well as a description of a system and a method. For the sake of simplicity, the terms “store”, “cause . . . to store”, or equivalent terms/phrases to these are used in the description, but these terms/phrases mean, when the functional units of the present embodiment are modules in a computer program, to cause a memory device to perform storage, or to control to cause a memory device to perform storage.

The functional units may correspond one-to-one to programs, but in implementation, one functional unit may be composed of one program, functional units may be composed of one program, or one module may be composed of programs. The functional units may be executed by a single computer, or a single functional unit may be executed by computers in a distributed or parallel environment. Note that one module may include other modules.

In addition to physical connections, the term “connection” is used below for logical connections (data transfer, instructions, reference relationships between data, logins, and so forth). The term “predetermined” means that it is determined prior to target processing, and is used to mean that it is determined not only before the process according to the present embodiment begins, but also even after the process according to the present embodiment begins, in accordance with the situation or state at that time or in accordance with the situation or state up until that time as long as it is before target processing. In a case where there is more than one “predetermined value”, each value may be different, or two or more values (including, of course, all values) may be identical.

The statement “in a case where A, do B” is used in the sense of “determine whether or not A is the case, and in a case where A is determined to be the case, do B”. Note that this excludes cases where a determination as to whether or not A is the case is unnecessary. In a case where things are enumerated, such as “A, B, C,” and so forth, it is an illustrative enumeration unless otherwise noted. The illustrative enumeration includes cases where only one of the things is selected (for example, only A).

A system or device is composed of computers, hardware devices, and devices, for example, that are connected by a network (including one-to-one communication connections) or other communication means, and also includes cases where the system or device is realized by a single computer, hardware device, or device, for example. The terms “device” and “system” are used synonymously with each other. As a matter of course, “system” does not include anything that is merely a social “mechanism” (a social system), which is an artificial arrangement.

For each process by each functional unit or for each process in a case where processes are performed within a functional unit, target information is read out from the memory device, and after the process, a processing result is written into the memory device. Thus, description regarding reading from the memory device before processing or writing to the memory device after processing may be omitted. Examples of the memory device in this case may include a hard disk, a random access memory (RAM), an external storage medium, a memory device connected via a communication line, and a register in the central processing unit (CPU).

As illustrated in FIG. 2A, the information processing system 10 includes an information processing device 100 and a measurement device 150.

The information processing device 100 determines whether the state related to the thinking of a user who is an examinee (also referred to simply as “state”) is a convergent thinking state, a divergent thinking state, or a relaxed state.

As illustrated in FIG. 2A, the information processing device 100 has, for example, an acquisition unit 110, a calculation unit 120, a determination unit 130, and a presentation unit 140.

The information processing device 100 may be a dedicated device or can be realized by executing a program on a general-purpose computer. The computer may be, for example, a desktop computer or a notebook computer, as well as a tablet, a smartphone, or a gaming device with interchangeable programs.

In a case where the information processing device 100 is included in a notebook computer, a tablet, a smartphone, or a gaming device, the device of interest may include the acquisition unit 110, the presentation unit 140, or the acquisition unit 110 and presentation unit 140.

The acquisition unit 110 may be configured to measure pieces of biometric information at once or may be configured to measure pieces of biometric information sequentially, with time differences, at specified times or in a predetermined order.

The measurement device 150 measures the user's biometric information. The measurement device 150 has a measurement unit 11, which is a sensor that measures the user's biometric information. The measurement device 150 inputs the user's measured biometric information to the acquisition unit 110. Examples of the sensor include an electrocardiographic sensor, a skin temperature sensor, and an electrodermal activity (EDA) sensor.

The skin temperature can be measured by a thermocouple attached to the user's middle finger with surgical tape. Skin temperature may be obtained by measuring the skin temperature of a part of the face (for example, forehead or nose) or a part of the body. Skin temperature may also be measured using a thermographic camera, an RGB camera, or an infrared camera.

There may be measurement devices 150, or there may be one measurement device 150. The number of measurement units 11 of the measurement device 150 may be two or more or may be one. There may be one acquisition unit 110, or there may be acquisition units 110.

The usage scenes for the information processing system 10 include, for example, offices, schools, tutoring schools, and homes, where work, study, reading, etc. are assumed, but are not limited to these scenes.

In order to obtain evaluation information values for the user's states (namely, the convergent thinking state, the divergent thinking state, and the relaxed state), for example, biometric information feature values, such as the coefficient of variation of R-R intervals (CVRR) and low frequency/high frequency (LF/HF), are extracted. The types or number of feature values are not specifically limited.

The information processing device 100 has, as its main hardware elements, a device having a processor that operates in accordance with programs and a device for an interface to connect external devices. The device having the processor may be a microprocessor or a microcontroller. The device for the interface has the function of connecting at least the calculation unit 120 and the determination unit 130. Desirably, the device for the interface has the function of performing data communication. The programs to be executed by the processor may be written in a read-only memory (ROM) in advance and then provided. Alternatively, the programs may be provided through telecommunication lines, such as the Internet, or on a computer-readable recording medium. The information processing system 10 can also be called a processing circuit. The information processing system 10 may also have a memory device.

The acquisition unit 110 is connected to the calculation unit 120. The acquisition unit 110 acquires the user's biometric information measured by the measurement device 150. In this case, the biometric information includes, for example, heart rate and pulse rate. Although this case is described as an example, the biometric information may also include electrodermal activity (EDA) and skin temperature. EDA may be skin potential level or skin potential reflection.

The calculation unit 120 is connected to the acquisition unit 110 and the determination unit 130. The calculation unit 120 acquires biometric information, such as heart rate variability, and calculates CVRR and LF/HF at a certain time. The calculation unit 120 causes the biometric information to include the calculated CVRR and LF/HF and provides the biometric information to the determination unit 130. The calculation unit 120 may receive the heart rate, pulse rate, skin temperature, and EDA from the acquisition unit 110 and provide the received heart rate, pulse rate, skin temperature, and EDA to the determination unit.

For example, the calculation unit 120 calculates CVRR from the heart rate intervals or pulse intervals. The calculation unit 120 also calculates LF/HF by performing, for a fixed time, FFT processing on the heart rate intervals or pulse intervals.

Note that the calculation unit 120 may calculate CVRR and LF/HF after the user executes a task or during the task execution.

The determination unit 130 is connected to the calculation unit 120. Furthermore, the determination unit 130 may be connected to the presentation unit 140. The determination unit 130 performs a determination process for determining, from the acquired biometric information, which of the convergent thinking state, divergent thinking state, or relaxed state corresponds to the user's thinking state.

Specifically, the determination unit 130 determines the user's state by determining whether or not CVRR and LF/HF exceed thresholds.

In the above-mentioned determination process, the determination unit 130 may determine that the user is in the convergent thinking state in a case where CVRR is less than a first threshold.

In the above-mentioned determination process, the determination unit 130 may determine that the user is in the divergent thinking state in a case where CVRR is greater than or equal to the first threshold and where LF/HF is greater than or equal to a second threshold.

In the above-mentioned determination process, the determination unit 130 may determine that the user is in the relaxed state in a case where CVRR is greater than or equal to the first threshold and where LF/HF is less than the second threshold.

Note that the determination unit 130 may make a determination after the user executes a task or during the task examination.

In this case, the user refers to a person who is the subject of measurement performed by the information processing device 100. Typically, the user is performing a task. Note that the user may be, for example, a person executing a task absentmindedly or a person who is meditating as a task.

The indicators used by the determination unit 130 to determine the user's state may be at least partially different for each determination. For example, different or the same indicators may be used as indicators for determining the user's state in the first half of the task and in the second half of the task.

Different thresholds may be used for the first half of the task and the second half of the task when determining the same user's state. For example, the user's state may be calculated by accumulating the time of a certain state of the user present during a unit time interval and dividing the accumulated time by the unit time to obtain a percentage.

The presentation unit 140 presents the user's state determined by the determination unit 130. The presentation unit 140 has, for example, a display device and presents the user's state by displaying an image indicating the user's state on a display screen (for example, a liquid crystal display). In this manner, the presentation unit 140 visualizes the user's state. The details presented by the presentation unit 140 will be described below.

The presentation unit 140 may present the user's state using light emitted from a lighting device, sound output by a sound device, or air conditioning performed by an air conditioning device, instead of displaying the user's state on a display. The presentation unit 140 may perform presentation after the user executes a task or during the task examination.

For example, the presentation unit 140 may retain, in a visualized state, the past plots on the graph indicating the user's state determined by the determination unit 130 to illustrate the trajectory of the transition of the user's state along with the current state. In addition to display on the display device, the presentation may include output of a three-dimensional (3D) image and may also be combined with, for example, printing performed by a printing device, such as a printer, sound output performed by an audio output device, such as a speaker, or vibration generated by a vibration generating device, such as a motor. The presentation unit 140 may be a general-purpose device, such as a smartphone, a tablet device, or a personal computer, or a gaming machine.

The measurement unit 11 and the measurement device 150 may be connected to each other through wireless communication, and the measurement device 150 and the information processing device 100 may be connected to each other through wireless communication.

The measurement unit 11 may be a heart rate monitor. The heart rate monitor measures the user's heart rate through a heart rate sensor that is, for example, affixed to the user's chest. The heart rate monitor may measure the user's heart rate through a non-contact sensor, such as a camera. The heart rate monitor may capture an image of the user's finger in close proximity to the camera and measure the user's pulse wave on the basis of changes in the skin chromaticity of the finger being captured. The camera may capture an image of the user's face and measure the user's pulse wave on the basis of changes in the skin chromaticity of the face. The heart rate monitor may measure, for example, pulse waves or blood pressure variations through a wristband (wristwatch) type pulse monitor or a pulse monitor clipped to the ear or finger. The heart rate monitor may be a device (also called a detector) that measures pulse waves by irradiating the user with irradiation light from a light source and detecting the reflected light generated due to the irradiation light.

The information processing device 100 accepts the user's biometric information provided by the measurement device 150 to determine the user's state. The information processing device 100 may present the determined result to the user. The presentation may be delivered in real time or collectively after the task is completed. The user who uses the information processing device 100 may be, for example, a researcher, the user's supervisor, or the user themselves. The person to whom the information processing device 100 presents the result of the determination may be the user or another user who uses the information processing device 100.

FIG. 2B is a flowchart illustrating a first example of processes performed by the information processing system 10 according to the present embodiment. The flowchart illustrated in FIG. 2B illustrates the procedure of the processes performed by the information processing system 10.

In Step S101, the acquisition unit 110 acquires biometric information from the measurement device 150.

In Step S102, the calculation unit 120 and the determination unit 130 determine the user's state.

In Step S103, the presentation unit 140 presents information regarding the user's state. Specifically, the presentation unit 140 presents the determination result obtained in Step S102 as the information regarding the user's state. The user's state may be presented in the form of a graph, such as a pie chart, line chart, or bar chart, or the user's state may also be presented using numbers or language. Note that the presentation unit 140 does not have to present information regarding the user's state.

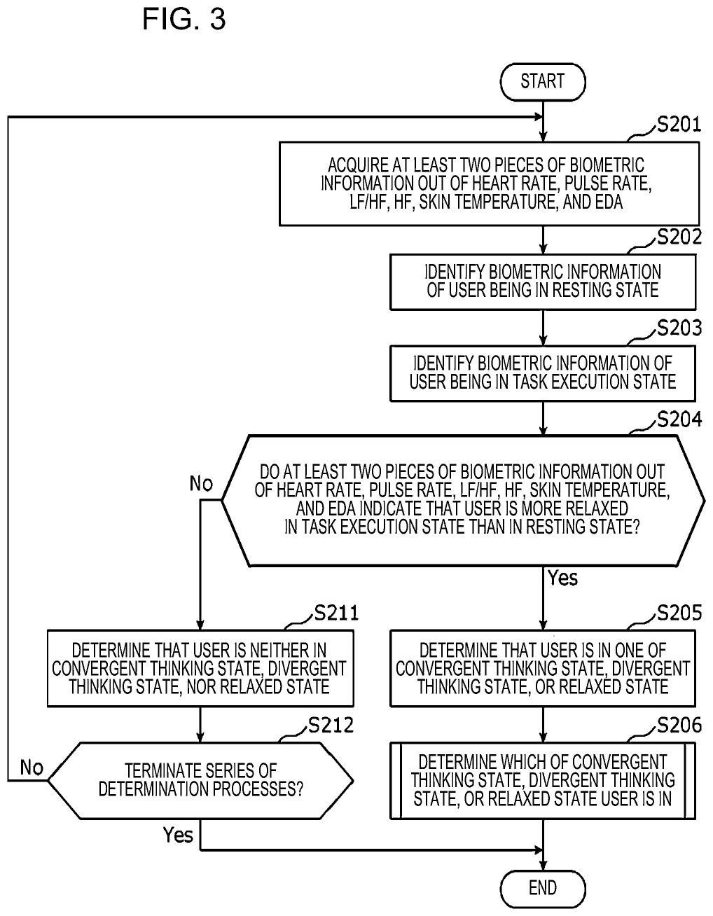

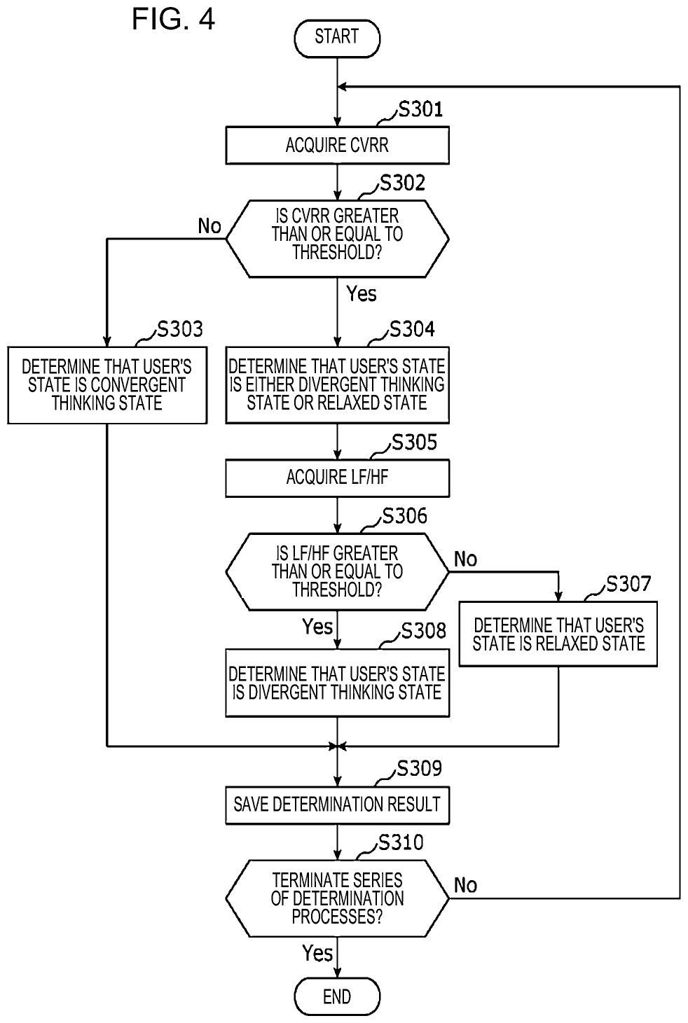

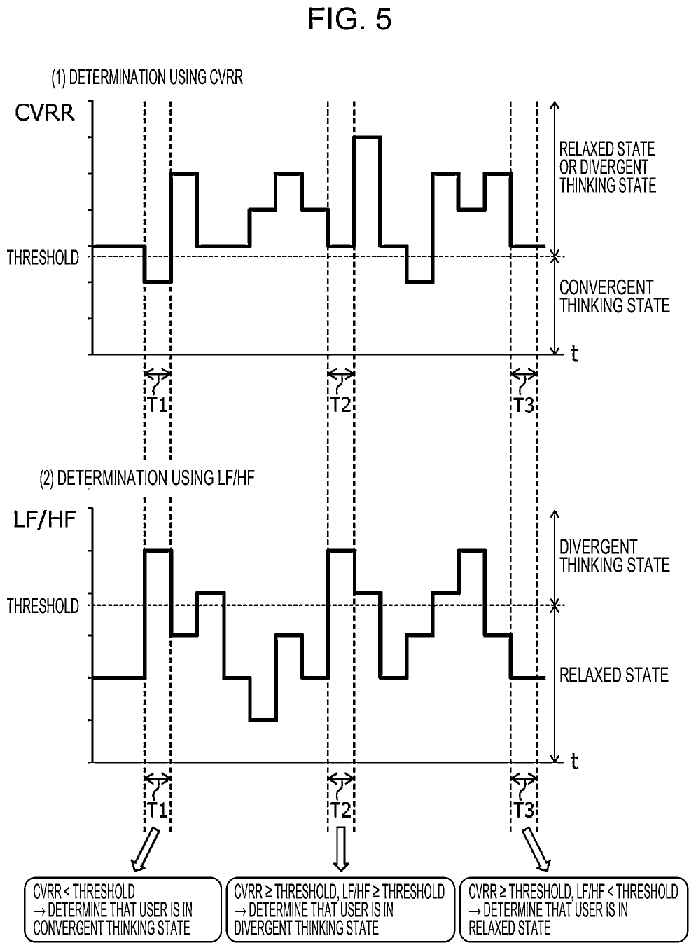

Hereinafter, with reference to FIGS. 3 and 4, the processes performed by the information processing system 10 will be specifically described.

FIG. 3 is a flowchart illustrating a second example of processes performed by the information processing system 10 according to the present embodiment.

FIG. 3 illustrates an example of processes performed by the information processing system 10 to determine which of the three states (the divergent thinking state, the convergent thinking state, or the relaxed state) corresponds to the user's state. Specifically, the information processing system 10 (a) performs a determination process (also referred to as a pre-determination process) to determine whether or not the user's state is one of the three states (the divergent thinking state, the convergent thinking state, or the relaxed state). In a case where the user's state is determined to be one of the three states in the pre-determination process, the information processing system 10 (b) performs a determination process to determine which of the three states (the divergent thinking state, the convergent thinking state, or the relaxed state) corresponds to the user's state. Note that the pre-determination process includes at least Steps S204, S205, and S211 (see below).

In Step S201, the measurement device 150 measures at least two out of the user's heart rate, pulse rate, LF/HF, HF, skin temperature, and EDA and provides them to the information processing device 100 as biometric information. The information processing device 100 (the acquisition unit 110) acquires the provided biometric information.

In Step S202, the determination unit 130 identifies the biometric information of the user being in a resting state from the biometric information acquired by the acquisition unit 110 in Step S201. The determination unit 130, for example, identifies the biometric information of the user being in the resting state by identifying, from the biometric information acquired by the acquisition unit 110 in Step S201, the biometric information included in a period in which the user is determined to be in the resting state. The period in which the user is determined to be in the resting state may be a different day than when the user performs the task or a different time of day than the time of day during which the user performs the task. That is, the time to measure the resting state may be scheduled immediately before the task, and the resting state may be measured. Alternatively, the time to measure the resting state may be scheduled on a different day than when the task is executed, and the resting state may be measured.

In Step S203, the determination unit 130 identifies the biometric information of the user being in a task execution state from the biometric information acquired by the acquisition unit 110 in Step S201. The determination unit 130, for example, identifies the biometric information of the user being in the task execution state by identifying, from the biometric information acquired by the acquisition unit 110 in Step S201, the biometric information included in a period in which the user is determined to be in the task execution state.

In Step S204, regarding the at least two pieces of biometric information out of the heart rate, pulse rate, LF/HF, HF, skin temperature, and EDA, the determination unit 130 determines whether (i) the values of the biometric information in the task execution state indicate that the user is more relaxed than the values of the biometric information in the resting state indicate or (ii) the values of the biometric information in the resting state and those in the task execution state are about the same.

Note that the at least two pieces of biometric information used in the above-mentioned determination are examples of at least N pieces of biometric information. A larger N has the advantage of increasing the accuracy of the determination that the user is relaxed. A smaller N allows for determining that the user is relaxed while reducing the processing amount of the processor.

For example, regarding heart rate, a relatively lower heart rate indicates that the user is relaxed. In a case where (the heart rate when the user is in the resting state)×(a first predetermined value)≥(the heart rate when the user is in the task execution state), it may be determined that the heart rate indicates that the user is relaxed. The first predetermined value may be determined using an appropriate number of examinees' heart rates. The first predetermined value may be less than 1.

Regarding LF/HF, a relatively lower LF/HF indicates that the user is relaxed. In a case where (the LF/HF determined on the basis of the heart rate obtained when the user is in the resting state)×(a second predetermined)≥(the LF/HF determined on the basis of the heart rate obtained when the user is in the task execution state), it may be determined that the LF/HF indicates that the user is relaxed. The second predetermined value may be determined using the LF/HF determined on the basis of an appropriate number of examinees' heart rates. The second predetermined value may be greater than 1.

Regarding HF, a relatively higher HF indicates that the user is relaxed. In a case where (the HF determined on the basis of the heart rate obtained when the user is in the resting state)×(a third predetermined value)≤(the HF determined on the basis of the heart rate obtained when the user is in the task execution state), it may be determined that the HF indicates that the user is relaxed. The third predetermined value may be determined using the HF determined on the basis of an appropriate number of examinees' heart rates. The third predetermined value may be less than 1.

Regarding skin temperature, a relatively higher skin temperature indicates that the user is relaxed. In a case where (the skin temperature when the user is in the resting state)×(a fourth predetermined value)≤(the skin temperature when the user is in the task execution state), it may be determined that the skin temperature indicates that the user is relaxed. The fourth predetermined value may be determined using an appropriate number of examinees' skin temperatures. The fourth predetermined value may be greater than 1.

Regarding EDA, relatively smaller fluctuations in the EDA value indicate that the user is relaxed. The above can be said about EDA because when you are feeling stress, the EDA value rises and then returns to its original value, and when you are not feeling stress, the above-mentioned changes in the EDA value do not occur. That is, in a case where (the amount of fluctuations of EDAs when the user is in the resting state)×(a fifth predetermined value)≥(the amount of fluctuations of EDAs when the user is in the task execution state), it may be determined that the EDA indicates that the user is relaxed. The fifth predetermined value may be determined using an appropriate number of examinees' EDAs. The fifth predetermined value may be less than 1.

Regarding pulse rate, a relatively lower pulse rate indicates that the user is relaxed. In a case where (the pulse rate when the user is in the resting state)×(a sixth predetermined value)≥(the pulse rate when the user is in the task execution state), it may be determined that the pulse rate indicates that the user is relaxed. The sixth predetermined value may be determined using an appropriate number of examinees' pulse rates. The sixth predetermined value may be less than 1.

The determination in S204 may be made on the basis of “at least two pieces of biometric information out of the heart rate, pulse rate, LF/HF, HF, skin temperature, EDA, brain waves, cerebral blood flow, facial expression, respiratory rate, myoelectric potential, and blood pressure”, instead of the “at least two pieces of biometric information out of the heart rate, pulse rate, LF/HF, HF, skin temperature, and EDA” described in S204.

In a case where the determination unit 130 determines that the values of the above-mentioned biometric information in the task execution state indicate that the user is more relaxed than the values of the biometric information in the resting state indicate, or the values of the biometric information in the resting state and those in the task execution state are about the same (Yes in Step S204), the process proceeds to Step S205. Otherwise (No in Step S204), the process proceeds to Step S211.

If Yes in S204, the process illustrated in Step S206 is performed. If No in S204, the process illustrated in Step S206 is not performed. This allows the processor to determine the user's state while reducing the amount of processing.

In Step S205, the determination unit 130 determines that the user is in one of the convergent thinking state, the divergent thinking state, or the relaxed state. The determination unit 130 may save the above-mentioned determination result therein.

In Step S206, the determination unit 130 determines which of the convergent thinking state, the divergent thinking state, or the relaxed state the user is in. Detailed processes included in Step S206 will be described later.

In Step S211, the determination unit 130 determines that the user is neither in the convergent thinking state, the divergent thinking state, nor the relaxed state. The determination unit 130 may save the above-mentioned determination result therein.

In Step S212, the determination unit 130 determines whether or not to terminate the series of determination processes illustrated in FIG. 3. For example, the determination unit 130 determines whether or not biometric information is provided from the calculation unit 120. In a case where it is determined that biometric information is no longer provided, the determination unit 130 may determine that the series of determination processes illustrated in FIG. 3 is to be terminated. Moreover, the determination unit 130 determines whether or not a preset time has elapsed. In a case where it is determined that the above-mentioned time has elapsed, the determination unit 130 may determine that the series of determination processes illustrated in FIG. 3 is to be terminated. By terminating the determination processes, the processing amount of the processor can be reduced.

In Step S212, in a case where it is determined that the series of determination processes illustrated in FIG. 3 is to be terminated (Yes in Step S212), the series of processes illustrated in FIG. 3 is terminated. Otherwise (No in Step S212), the process proceeds to Step S201. In this manner, the information processing device 100 repeatedly performs the above-mentioned determination processes until the information processing device 100 determines that the series of determination processes illustrated in FIG. 3 is to be terminated.

FIG. 4 is a flowchart illustrating a third example of processes performed by the information processing system 10 according to the present embodiment. The processes illustrated in FIG. 4 are performed by the determination unit 130 and details the processes included in Step S206 of FIG. 3.

In Step S301, the determination unit 130 acquires CVRR from the calculation unit 120. The CVRR that the determination unit 130 acquires is CVRR calculated by the calculation unit 120 from the electrocardiogram waveform acquired by the acquisition unit 110.

In Step S302, it is determined whether or not the CVRR acquired in Step S201 is greater than or equal to a threshold. In a case where it is determined that the CVRR is greater than or equal to the threshold (Yes in Step S302), the process proceeds to Step S304. Otherwise (No in Step S302), the process proceeds to Step S303. The determination process in Step S302 is also referred to as a first determination process.

The threshold for CVRR used in Step S302 is included in the range of 3.5% to 4.2%, for example. In an experiment conducted in advance, the result was obtained that the mean CVRR of users performing a task of inducing users to the convergent thinking state was 3.43%, and the mean CVRR of the users performing a task of inducing users to the divergent thinking state was 4.29%. Therefore, by using a threshold within the above-mentioned range as a threshold between 3.43% and 4.29%, it is possible to appropriately determine whether the user is in the convergent thinking state or the divergent thinking state.

In Step S303, the determination unit 130 determines that the user's state is the convergent thinking state.

In Step S304, the determination unit 130 determines that the user's state is either the divergent thinking state or the relaxed state.

In Step S305, the determination unit 130 acquires LF/HF from the calculation unit 120. The LF/HF that the determination unit 130 acquires is the LF/HF calculated by the calculation unit 120 from the electrocardiogram waveform acquired by the acquisition unit 110.

In Step S306, it is determined whether the LF/HF acquired in Step S305 is greater than or equal to a threshold. In a case where it is determined that the LF/HF is greater than or equal to the threshold (Yes in Step S306), the process proceeds to Step S308. Otherwise (No in Step S306), the process proceeds to Step S307. The determination process in Step S306 is also referred to as a second determination process.

The threshold for LF/HF used in Step S306 is included in the range of 3.0% to 4.5%, for example. In an experiment conducted in advance, the result was obtained that the mean LF/HF of users performing the task of inducing users to the convergent thinking state was 2.57%, and the mean CVRR of the users performing the task of inducing users to the divergent thinking state was 4.91%. Therefore, by using a threshold within the above-mentioned range as a threshold between 2.57% and 4.91%, it is possible to appropriately determine whether the user is in the divergent thinking state or the relaxed state.

In Step S307, the determination unit 130 determines that the user's state is the relaxed state.

In Step S308, the determination unit 130 determines that the user's state is the divergent thinking state.

In Step S309, the determination unit 130 saves the determination result regarding the user's state. Specifically, the determination unit 130 saves the determination result in Step S303, Step S307, or Step S308. Note that the determination process in Steps S303, S307, and S308 is also referred to as a third determination process.

In Step S310, the determination unit 130 determines whether or not to terminate the series of determination processes illustrated in FIG. 4. For example, the determination unit 130 determines whether or not CVRR or LF/HF is provided from the calculation unit 120. In a case where it is determined that one of or both CVRR and LF/HF are no longer provided, the determination unit 130 may determine that the series of determination processes illustrated in FIG. 4 is to be terminated. Moreover, the determination unit 130 determines whether or not a preset time has elapsed. In a case where it is determined that the above-mentioned time has elapsed, the determination unit 130 may determine that the series of determination processes illustrated in FIG. 4 is to be terminated.

In Step S310, in a case where it is determined that the series of determination processes illustrated in FIG. 4 is to be terminated (Yes in Step S310), the series of processes illustrated in FIG. 4 is terminated. Otherwise (No in Step S310), the process proceeds to Step S301. In this manner, the information processing device 100 repeatedly performs the above-mentioned determination processes until the information processing device 100 determines that the series of determination processes illustrated in FIG. 4 is to be terminated.

FIG. 5 includes diagrams for describing examples of states determined by the information processing system 10 according to the present embodiment.

FIG. 5 includes diagrams in which the result of the user's state determination made by the determination unit 130 (Step S102), namely, whether the user is in the convergent thinking state, the divergent thinking state, or the relaxed state is illustrated on a time axis.
(1) of FIG. 5 illustrates the mean CVRR values of the user for each time segment and a threshold for CVRR.
(2) of FIG. 5 illustrates the mean LF/HF values of the user for each time segment and a threshold for LF/HF.

In a time segment T1 illustrated in FIG. 5, CVRR is less than the threshold. The determination unit 130 thus determines that the user is in the convergent thinking state (Step S303).

In a time segment T2 illustrated in FIG. 5, CVRR is greater than or equal to the threshold, and LF/HF is greater than or equal to the threshold. The determination unit 130 thus determines that the user is in the divergent thinking state (Step S308).

In a time segment T3 illustrated in FIG. 5, CVRR is greater than or equal to the threshold, and LF/HF is less than the threshold. The determination unit 130 thus determines that the user is in the relaxed state (Step S307).

In a case where the user performs a complex task, it takes some time from the time when the task is presented to the user until the time when the user understands what the task is and begins to perform the task. Thus, in a case where the determination unit 130 determines the user's state, the determination unit 130 may use the user's state at a certain time after the user starts the task.

Furthermore, by considering that changes in the user's states occur a certain time after a task, in order to determine these states in association with the task, it is also permissible to treat, as a single determination target, a time segment that includes a certain duration after each task's end time for each identical task.

In the determination process illustrated in FIG. 4, for example, threshold information for determining the value to which the threshold is to be set is stored in the memory device, and a threshold set using the threshold information acquired from the memory device may be used.

The threshold information may be stored in association with an ID for identifying an individual. In this case, the threshold appropriate for the individual can be used to determine a state related to thinking, thereby improving the accuracy of the determination.

The ID-associated threshold information may be a value that takes into account CVRR and LF/HF of the individual being in the resting state. The CVRR and LF/HF of each individual user being in the resting state may be stored in association with the ID in the memory device, and the threshold information for each individual user may be generated. The resting state may be a neutral state (in other words, a neutral state in terms of thinking) induced by having the user listen to white noise or utter meaningless words. Alternatively, the resting state may be a state in which the user is not engaged in a task or other activity. The threshold information may be updated on the basis of state determination results.

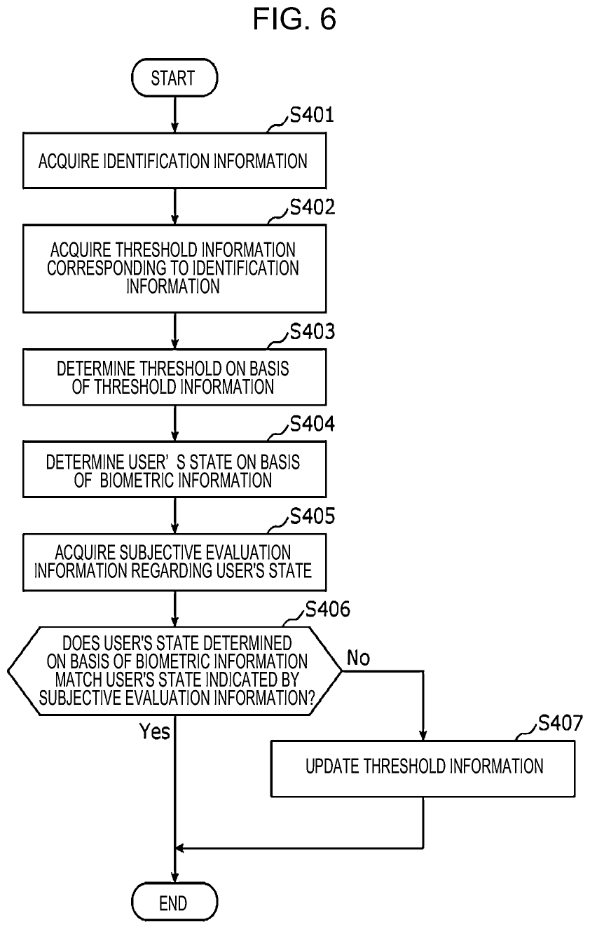

FIG. 6 is a flowchart illustrating an example of a process regarding the update of threshold information according to the present embodiment. FIG. 6 illustrates a process for making a determination using the threshold information associated with the user's identification information.

In Step S401, the determination unit 130 acquires the user's identification information. The identification information is information that can uniquely identify the user. The identification information may be letters or symbols entered by the user using input devices, such as a keyboard, or may be an image of the user's face captured by a camera.

In Step S402, the determination unit 130 acquires, from the memory device, the threshold information corresponding to the identification information acquired in Step S401.

In Step S403, the determination unit 130 determines a threshold on the basis of the threshold information acquired in Step S402. The threshold to be determined is a threshold for CVRR (corresponding to the first threshold), or both the threshold for CVRR (corresponding to the first threshold) and the threshold for LF/HF (corresponding to the second threshold) may be determined.

In Step S404, the determination unit 130 performs a determination process based on the user's biometric information. The determination process performed in Step S404 is the processes illustrated in FIGS. 3 and 4.

In Step S405, the determination unit 130 acquires subjective evaluation information regarding the user's state. The subjective evaluation information corresponds to the user's state evaluated by the user themselves. The determination unit 130 causes a graphical user interface (GUI) to be presented. The GUI includes, for example, a message asking about the user's state during the task such as “Please self-evaluate your state during the task.” and options for the user's state during the task (specifically, “divergent thinking state”, “relaxed state”, and “convergent thinking state”) and accepts a response from the user. In a case where the user responds with “divergent thinking state” using the GUI, the information representing that the user was in the divergent thinking state while performing the task is acquired as the subjective evaluation information. Note that the information acquired in Step S405 is not limited to the subjective evaluation information obtained as a result of evaluation performed by the user themselves but may also be evaluation information indicating a determination result determined using a method different from the method used to determine the state based on heart rate. In this case, the case where the evaluation information is subjective evaluation information will be described as an example.

In Step S406, the determination unit 130 determines whether or not the user's state determined in Step S404 matches the user's state indicated by the subjective evaluation information acquired in Step S405. For example, in a case where the user's state determined on the basis of the biometric information is the convergent thinking state and where the user's state indicated by the subjective evaluation information is the convergent thinking state, the determination unit 130 determines that these states match. In contrast, for example, in a case where the user's state determined on the basis of the biometric information is the convergent thinking state and where the user's state indicated by the subjective evaluation information is the divergent thinking state, the determination unit 130 determines that these states do not match.

In a case where these states match (Yes in Step S406), the series of processes illustrated in FIG. 6 is terminated. Otherwise (No in Step S406), the process proceeds to Step S407.

In Step S407, the determination unit 130 updates the threshold information associated with the user (namely, the identification information) on the basis of the user's determined state and the user's state indicated by the subjective evaluation information. For example, in a case where the user's state determined on the basis of the biometric information is the convergent thinking state and where the user's state indicated by the subjective evaluation information is the divergent thinking state, the threshold information is updated so as to lower the CVRR threshold indicated by the threshold information. This is because lowering the CVRR threshold can expand the CVRR range that is determined to be greater than or equal to the threshold (Step S302) and contribute to determining the user's state to be the divergent thinking state.

The same applies to the updating of the LF/HF threshold, and a detailed description will be omitted.

As described above, the accuracy of state determinations can be improved by updating the threshold information using the results of subjective evaluations.

In the following, the presentation of information regarding the user's state will be described. The information to be presented includes the determination result saved in Step S309 of FIG. 4 and is presented to the user using the presentation unit 140.

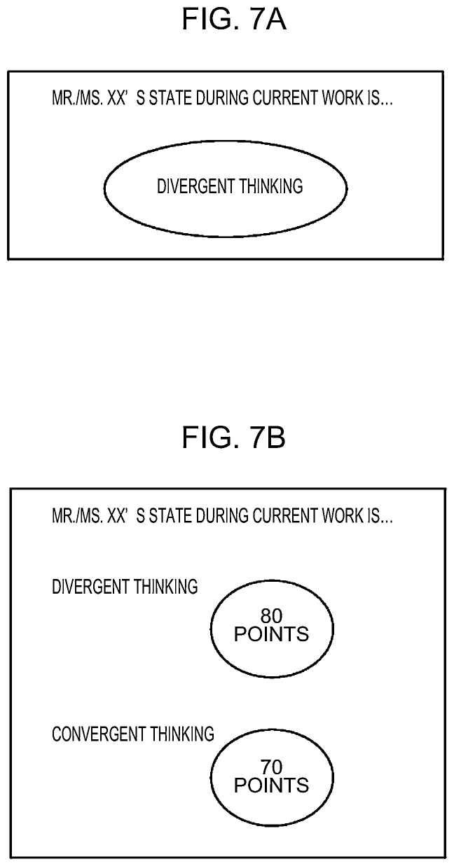

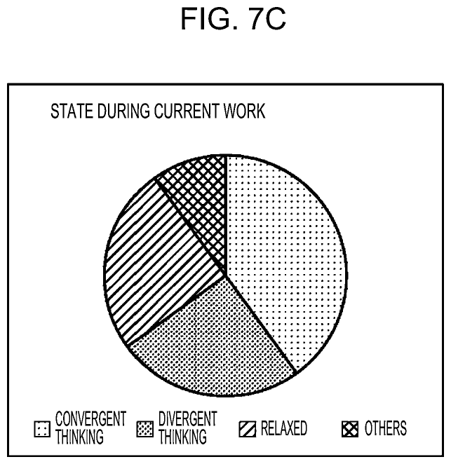

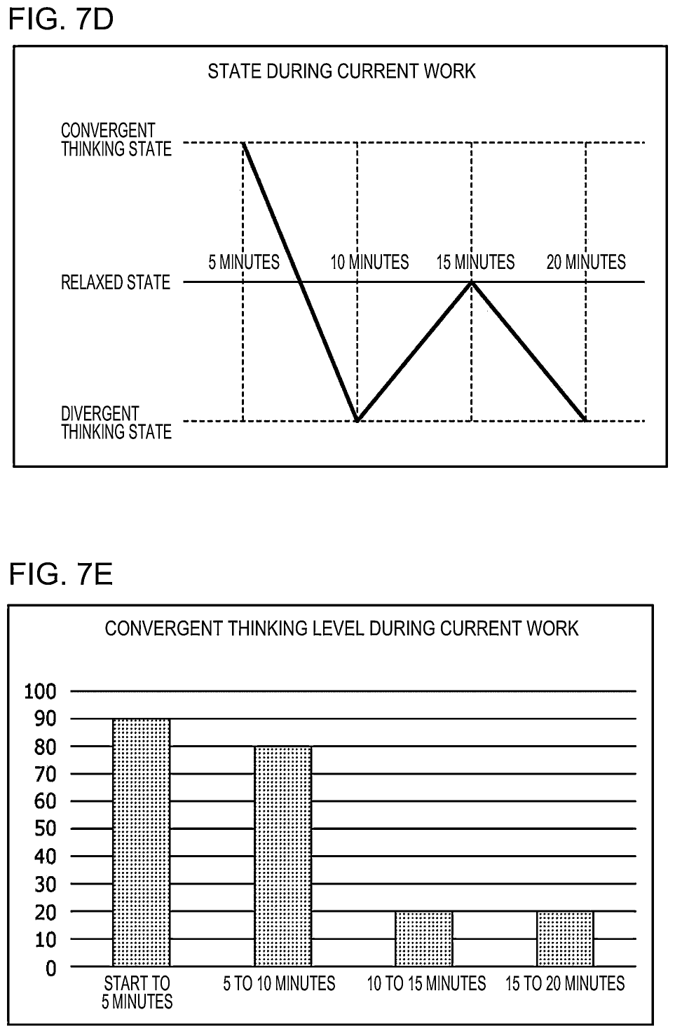

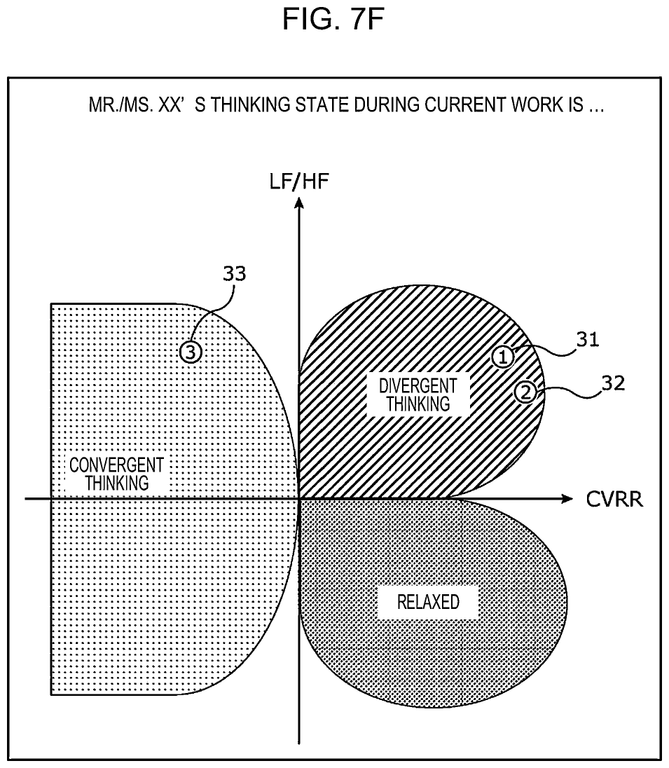

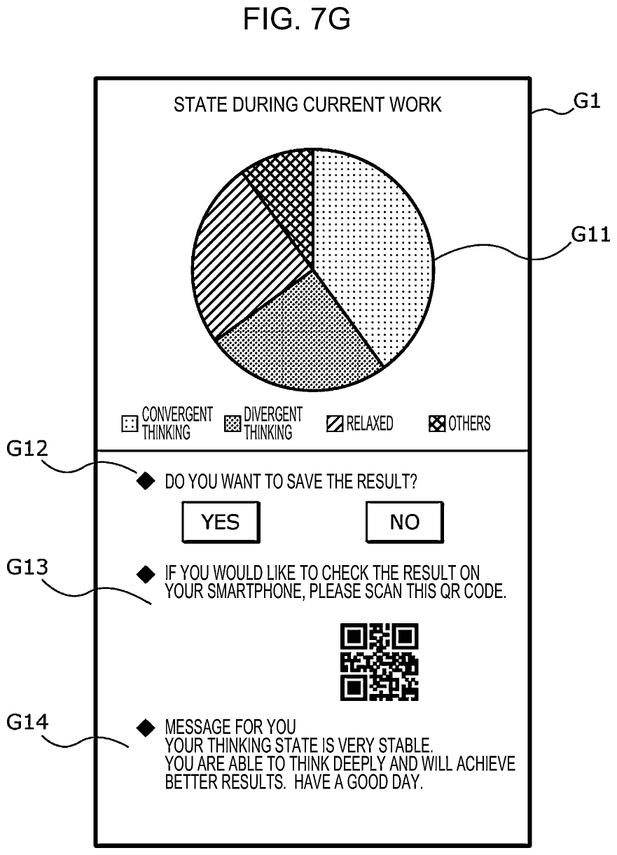

The diagram (for example, a graph) illustrating the determination results presented by the presentation unit 140 may illustrate the user's state (namely, the convergent thinking state, the divergent thinking state, or the relaxed state) in time series (FIG. 7D) or may illustrate percentages or ratios of the user's state(s) (FIG. 7C or 7E). The diagram may illustrate the user's state or states only (FIG. 7A, 7C, 7D, or 7F) or may illustrate the user's state together with its determined evaluation result or the user's states together with their determined evaluation values (FIG. 7B or 7E). Each evaluation value may be a value that indicates the level of validity as the user's state, a value that indicates the level of stability in thinking based on the time period during which a certain state has been maintained continuously, or a value that indicates the percentage of the time period of a certain state in the duration of the task. In a case where the evaluation value is displayed, the evaluation value may be displayed as a score, with the convergent thinking state receiving x points, the divergent thinking state y points, or the relaxed state z points (FIG. 7B). More general expressions may be used to describe the convergent thinking state as “focused” and the divergent thinking state as “creative”. The determination result may be an evaluation determination result of the entire business hours or may be a determination result of a state in a specific time period.

In a case where the determination unit 130 determines the user's state and thereafter converts the determined state into a score, the score may be determined on the basis of the amount of change from the user's biometric information acquired in advance in the resting state to the biometric information in the task execution state. The score may be calculated by comparing the mean value of the biometric information measured in the user's task execution state with the mean feature value of the data of persons acquired in prior experiments. Furthermore, the score may be calculated by comparing the calculated amount of change from the user's biometric information acquired in advance in the resting state to the biometric information in the task execution state with the mean variation of the feature data of persons acquired in prior experiments.

Examples of the presentation of the determination results according to the present embodiment will be illustrated in FIGS. 7A to 7G.

FIG. 7A is a diagram for describing a first example of the presentation of a determination result according to the present embodiment. FIG. 7A is an example in which the trend of the user's state during work is presented using words. In FIG. 7A, “divergent thinking” is an abbreviation for “divergent thinking state”. The abbreviation for “convergent thinking state” can be “convergent thinking”, and the abbreviation for “relaxed state” can be “relaxed”. The same will apply thereafter. Note that the presentation in FIG. 7A is not limited to display on the screen, but may also be presented via audio.

FIG. 7B is a diagram for describing a second example of the presentation of a determination result according to the present embodiment. FIG. 7B is an example in which the determination result of the user's states during work is presented in the form of a score. The score presented may be only a score for a specific state, or the scores for states may be presented. The maximum score may be 100 points or may be 5 points, for example.

FIG. 7C is a diagram for describing a third example of the presentation of a determination result according to the present embodiment. FIG. 7C illustrates an example of a pie chart illustrating the percentages of the user's states (namely, the convergent thinking state, the divergent thinking state, and the relaxed state) during work.

FIG. 7D is a diagram for describing a fourth example of the presentation of determination results according to the present embodiment. FIG. 7D is an example in which the user's state during work is illustrated in time series. The horizontal axis represents time, and the vertical axis represents the user's state (namely, the convergent thinking state, the divergent thinking state, or the relaxed state). Note that the time indicated by the horizontal axis may indicate a single point in time or may indicate several minutes of time.

FIG. 7E is a diagram for describing a fifth example of the presentation of determination results according to the present embodiment. FIG. 7E is an example of the user's convergent thinking state score (also referred to as convergent thinking level) during work in time series. The horizontal axis represents time, and the vertical axis represents the user's convergent thinking state score. Note that the vertical axis may represent the user's divergent thinking state score or the user's relaxed state score. The results of thinking states may also be displayed in a collective manner.

FIG. 7F is a diagram for describing a sixth example of the presentation of

determination results according to the present embodiment. FIG. 7F illustrates an example in which the user's states during work are indicated with circular marks on a coordinate plane composed of a horizontal axis representing CVRR and a vertical axis representing LF/HF. In FIG. 7F, the first quadrant indicates the divergent thinking state, the second and third quadrants indicate the convergent thinking state, and the fourth quadrant indicates the relaxed state.

For example, a mark 31 illustrated in FIG. 7F indicates the user's state five minutes after the start of the work and specifically indicates that the user's state five minutes after the start of the work is the divergent thinking state. A mark 32 illustrated in FIG. 7F indicates the user's state ten minutes after the start of the work and specifically indicates that the user's state ten minutes after the start of the work is the divergent thinking state. A mark 33 illustrated in FIG. 7F indicates the user's state 15 minutes after the start of the work and specifically indicates that the user's state 15 minutes after the start of the work is the convergent thinking state.

The labels on the horizontal axis and vertical axis may be CVRR and LF/HF, respectively, or other words. As marks illustrated in FIG. 7F, points indicating the states of respective time segments during work may be illustrated, or a single point indicating the average state of the entire work may be illustrated.

FIG. 7G is a diagram for describing a seventh example of the presentation of a determination result according to the present embodiment. FIG. 7G is a diagram illustrating an example of presentation including information other than a determination result. FIG. 7G is a diagram illustrating an example of a display image G1 displayed on the presentation unit 140 as presentation information. The display image G1 includes a graph G11, an input field G12, and message display areas G13 and G14.

The graph G11 is substantially the same as the graph illustrated in FIG. 7C.

The input field G12 is a field where the user selects and inputs whether or not to save the determination result. The input field G12 displays the message “Do you want to save the result?”, a “YES” button, and a “NO” button. The “YES” button is for the user to select to save the determination result. The “NO” button is for the user to select not to save the determination result. When the user operates to select the “YES” button, the presentation unit 140 accepts the operation and changes the color of the “YES” button. When the user operates to select the “NO” button, the presentation unit 140 accepts the operation and changes the color of the “NO” button. This allows the user to easily confirm which button is selected.

When the user operates to select the “YES” button, the presentation unit 140 accepts the operation, saving the determination result is “permitted”, and the determination result regarding the user's state is saved. When the user operates to select the “NO” button, the presentation unit 140 accepts the operation, saving the determination results is “not permitted”, the determination result regarding the user's state is not saved. Since the determination result for “not permitted” is not saved in a memory unit, unnecessary memory consumption can be reduced.

The message display area G13 includes a QR code®. The message display area G13 also includes a message to notify the user that the user can check the result from this instance on their mobile terminal by separately scanning the QR code. In FIG. 7G, the message display area G13 includes the message “If you would like to check the result on your smartphone, please scan this QR code.” and the QR code. Although the example illustrates a QR code, the user's e-mail address may be entered. Moreover, a card reader may be attached to the presentation unit 140, and in a case where a card, such as an employee ID card, is read by the card reader, the determination result may be transmitted to the user and their supervisor, for example.

The message display area G14 includes a message that takes into account the user's biometric information during work and the climate and time of day. In FIG. 7G, assuming a user who has been in a specific thinking state for a long time in the early hours of the morning, the positive message “Your thinking state is very stable. You are able to think deeply and will achieve better results. Have a good day.” is displayed.

Next, the process performed by the information processing device 100 to control lighting in accordance with determination results will be described.

The information processing device 100 compares a target state targeted by the user with the state related to the user's thinking as determined by the information processing device 100. In a case where, as a result of the comparison, the target state is different from the determined state related to the user's thinking, the information processing device 100 performs control to change the characteristics of illuminating light emitted by a lighting device that illuminates a surrounding area of the user. The control of the lighting device will be described in more detail below.

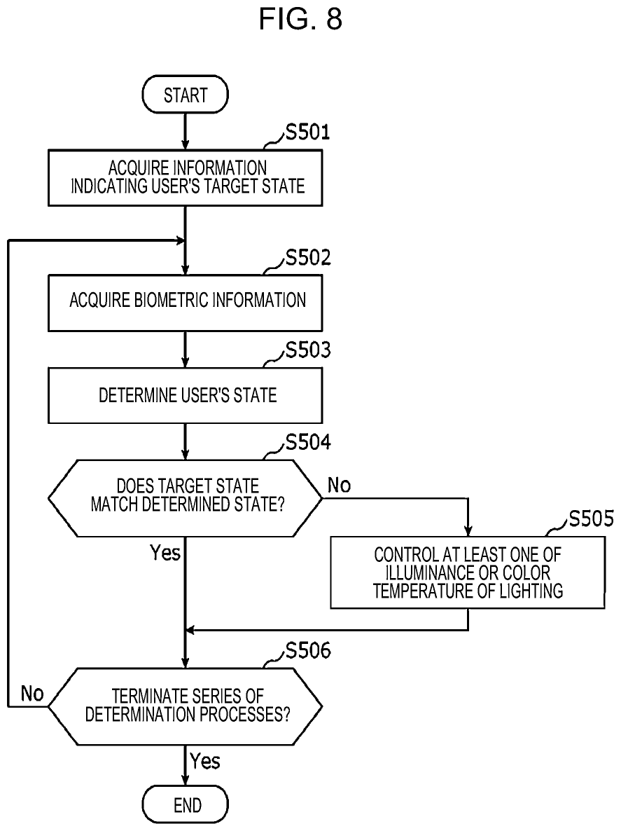

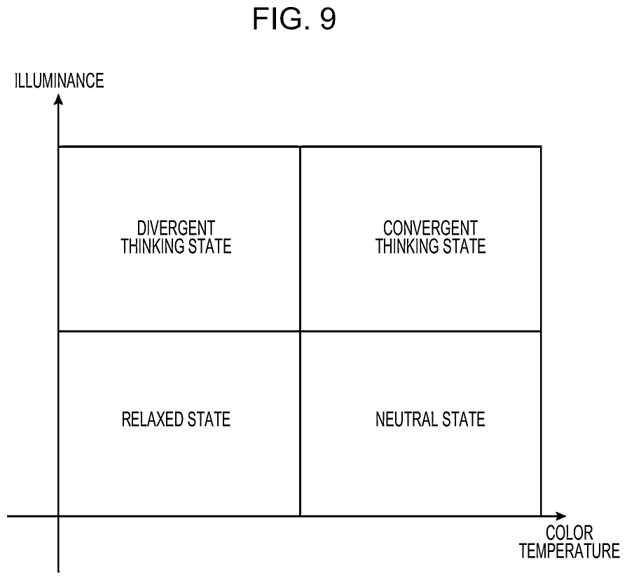

FIG. 8 is a flowchart illustrating an example of a process for controlling lighting in accordance with a determination result according to the present embodiment.

In Step S501, the acquisition unit 110 acquires information regarding the user's target state.

The processes in Steps S502 to S503 are substantially the same as the processes in Steps S101 to S102 in FIG. 2B.

In Step S504, the determination unit 130 determines whether or not the target state acquired by the acquisition unit 110 in Step S501 matches the state determined in Step S503. In a case where it is determined that these states match (Yes in Step S504), the process proceeds to Step S506. In a case where these states do not match (No in Step S504), the process proceeds to Step S505.

In Step S505, the presentation unit 140 controls at least one of the illuminance or color temperature of the lighting. Details of the control will be described below.

In Step S506, the determination unit 130 determines whether or not to terminate the series of determination processes illustrated in FIG. 8. For example, the determination unit 130 determines whether or not the acquisition of new biometric information (Step S502) is successful. In a case where the acquisition of new biometric information is no longer successful, the determination unit 130 may determine that the series of determination processes illustrated in FIG. 8 is to be terminated. Moreover, the determination unit 130 determines whether or not a preset time has elapsed. In a case where it is determined that the above-mentioned time has elapsed, the determination unit 130 may determine that the series of determination processes illustrated in FIG. 8 is to be terminated. By terminating the determination processes, the processing amount of the processor can be reduced.

FIG. 9 is a diagram illustrating the relationship between the illuminance and color temperature of lighting suitable for inducing the user to a certain state (namely, the convergent thinking state, the divergent thinking state, or the relaxed state). The relationship between the illuminance and color temperature of such lighting is described in the work by Koichiro FUMOTO, Yutaka HASHIURA, Kouzou TSUJI, and Takeki KIMURA titled “Office lighting system to encourage creativity of workers-Study on optimal lighting conditions for creative work in the office-,” presented in Japan Human Factors and Ergonomics Society Kansai Branch Rombun-shu 2009, pages 171-174, Dec. 5, 2009.

The information processing device 100 can thus induce the user to the divergent thinking state, the convergent thinking state, or the relaxed state by controlling the lighting device, as one example.

### (1) Control to Induce User to Divergent Thinking State

For example, the use of lighting with high illuminance and low color temperature as conditions suitable for work such as brainstorming, namely, conditions suitable for the divergent thinking state is described in the work by Koichiro FUMOTO, Yutaka HASHIURA, Kouzou TSUJI, and Takeki KIMURA titled “Office lighting system to encourage creativity of workers-Study on optimal lighting conditions for creative work in the office-,” presented in Japan Human Factors and Ergonomics Society Kansai Branch Rombun-shu 2009, pages 171-174, Dec. 5, 2009.

In contrast, when it is desired to induce the user to the divergent thinking state, the user's state is presumed to be different from the divergent thinking state and to be the convergent thinking state or the relaxed state, for example.

In this case, in a case where the information processing device 100 controls the lighting device to lower the color temperature of the illuminating light and increase the illuminance of the illuminating light, it may be possible to induce the user to the divergent thinking state (see FIG. 9).

Thus, in a case where the user's CVRR is determined to be less than the threshold (that is, the user is determined to be in the convergent thinking state) or in a case where the user's CVRR is determined to be greater than or equal to the threshold and the LF/HF is determined to be less than the threshold (that is, the user is determined to be in the relaxed state), when the information processing device 100 controls the lighting device to lower the color temperature of the illuminating light and increase the illuminance of the illuminating light, it may be possible to induce the user to the divergent thinking state.

### (2) Control to Induce User to Convergent Thinking State

For example, the use of lighting with high color temperature and high illuminance as conditions suitable for the convergent thinking state is described in the work by Koichiro FUMOTO, Yutaka HASHIURA, Kouzou TSUJI, and Takeki KIMURA titled “Office lighting system to encourage creativity of workers-Study on optimal lighting conditions for creative work in the office-,” presented in Japan Human Factors and Ergonomics Society Kansai Branch Rombun-shu 2009, pages 171-174, Dec. 5, 2009.

In contrast, when it is desired to induce the user to the convergent thinking state, the user's state is presumed to be different from the convergent thinking state and to be the divergent thinking state or the relaxed state, for example.

In this case, in a case where the information processing device 100 controls the lighting device to increase the color temperature of the illuminating light and increase the illuminance of the illuminating light, it may be possible to induce the user to the convergent thinking state (see FIG. 9).

Thus, in a case where the user's CVRR is determined to be greater than or equal to the threshold and the LF/HF is determined to be greater than or equal to the threshold (that is, the user is determined to be in the divergent thinking state) or in a case where the user's CVRR is determined to be greater than or equal to the threshold and the LF/HF is determined to be less than the threshold (that is, the user is determined to be in the relaxed state), when the information processing device 100 controls the lighting device to increase the color temperature of the illuminating light and increase the illuminance of the illuminating light, it may be possible to induce the user to the convergent thinking state.

### (3) Control to Induce User to Relaxed State

For example, the use of lighting with low color temperature and low illuminance as conditions suitable for the relaxed state is described in the work by Koichiro FUMOTO, Yutaka HASHIURA, Kouzou TSUJI, and Takeki KIMURA titled “Office lighting system to encourage creativity of workers-Study on optimal lighting conditions for creative work in the office-,” presented in Japan Human Factors and Ergonomics Society Kansai Branch Rombun-shu 2009, pages 171-174, Dec. 5, 2009.

In contrast, when it is desired to induce the user to the relaxed state, the user's state is presumed to be different from the relaxed state and to be the divergent thinking state or the convergent thinking state, for example.

In this case, in a case where the information processing device 100 controls the lighting device to lower the color temperature of the illuminating light and lower the illuminance of the illuminating light, it may be possible to induce the user to the relaxed state (see FIG. 9).

Thus, in a case where the user's CVRR is determined to be less than the threshold (that is, the user is determined to be in the convergent thinking state) or in a case where the user's CVRR is determined to be greater than or equal to the threshold and the LF/HF is determined to be greater than or equal to the threshold (that is, the user is determined to be in the divergent thinking state), when the information processing device 100 controls the lighting device to lower the color temperature of the illuminating light and lower the illuminance of the illuminating light, it may be possible to induce the user to the relaxed state.

As described above, the information processing device 100 according to the present embodiment can determine the state related to the user's thinking with higher accuracy.

In the embodiment described above, each structural element may be configured using dedicated hardware or may be realized by executing a software program suitable for each structural element. Each structural element may be realized by a program execution unit, such as a central processing unit (CPU) or processor, reading out and executing a software program recorded on a recording medium, such as a hard disk or semiconductor memory. In this case, the software that realizes the information processing device, for example, according to the embodiment described above is a program as follows.

That is, this program is a program that causes a computer to perform an information processing method including acquisition of the user's CVRR and LF/HF through a sensor and performance of the determination process for determining, using a processor, which of the convergent thinking state, the divergent thinking state, or the relaxed state corresponds to the state related to the user's thinking on the basis of the acquired CVRR and LF/HF.

The configuration may also be such that one of the CPU and processor, which perform the thinking state determination process, and the memory device is provided in a server that communicates via a network with a sensor that detects the user's biometric information.

As described above, the information processing methods according to, for example, one or more aspects have been described on the basis of the embodiment; however, the present disclosure is not limited to this embodiment. Forms obtained by adding various changes that one skilled in the art can conceive of to the present embodiment, as well as forms constructed by combining structural elements in different embodiments, may also be included in the scope of one or more aspects, as long as these forms do not depart from the gist of the present disclosure.

### Other 1

Modifications of the embodiment of the present disclosure may be those described below.

A method according to a first item is
a method performed by a processor,
the method including:
performing, using the processor, a first process, the first process not including a frequency analysis process, and
performing, using the processor, a second process in a case where a result of the first process satisfies a predetermined condition, the processor not performing the second process in a case where the result of the first process does not satisfy the predetermined condition,
the first process is (i) a first determination process, (ii) a second determination process, (iii) a third determination process, (iv) the first determination process and second determination process, (v) the first determination process and third determination process, (vi) the second determination process and third determination process, or (vii) the first determination process, second determination process, and third determination process,
the first determination process is to determine whether or not a heart rate of a user is smaller than a first threshold,
the second determination process is to determine whether or not a skin temperature of the user is smaller than a second threshold,
the third determination process is to determine whether or not a difference between a maximum electrodermal activity of the user and a minimum electrodermal activity of the user in a predetermined period is smaller than a third threshold, and
the second process includes
calculating CVRR on the basis of a first standard deviation of heart rate intervals of the user and a first mean of the heart rate intervals,
performing frequency analysis on the heart rate intervals to obtain a power spectrum density function of the heart rate intervals of the user,
calculating LF/HF on the basis of a first integral value of a first section of the power spectrum density function and a second integral value of a second section of the power spectrum density function,
determining a state related to the user on the basis of the CVRR and the LF/HF, and
outputting the determined state.

The method described above indicates that the processor does not perform the second process in a case where the result of the first process does not satisfy the predetermined condition. That is, in a case where the result of the first process does not satisfy the predetermined condition, the processor performs neither the frequency analysis process, a standard deviation calculation process, a mean calculation process, nor an integral value calculation process, and thereby the processor can determine the user's state while reducing the amount of processing.

The amount of processing obtained by adding the amount of processing for the first determination process, that for the second determination process, and that for the third determination process is less than the amount of processing for the process. As a result, the processor can determine the user's state while reducing the amount of processing.

The frequency analysis process may be a process in which a fast Fourier transform is performed.

The first threshold may be (a heart rate in a case where the user is in a resting state)×a, where a may be greater than 1.

The second threshold may be (a skin temperature in a case where the user is in a resting state)×b, where b may be greater than 1.

The third threshold may be {(a maximum electrodermal activity included in electrodermal activities in a case where the user is in a resting state)−(a minimum electrodermal activity included in electrodermal activities in a case where the user is in a resting state)}×c, where c may be greater than 1.

A method according to a second item is the method according to the first item, in which
the CVRR is (the first standard deviation)/(the first mean),
the LF/HF is (the first integral value)/(the second integral value),
a beginning of the first section is a first frequency,
an end of the first section is a second frequency,
a beginning of the second section is a third frequency,
an end of the second section is a fourth frequency,
the first frequency is lower than the second frequency,
the third frequency is lower than the fourth frequency,
the first frequency is lower than the third frequency, and
the second frequency is lower than the fourth frequency.

A method according to a third item is the method according to the second item, in which
an electrocardiogram waveform of the user is measured using electrodes,
the heart rate intervals are determined on the basis of the electrocardiogram waveform,
each of the heart rate intervals is a time interval between adjacent R waves in the electrocardiogram waveform,
the skin temperature is measured using a thermocouple or a camera, and
the electrodermal activities are measured using electrodes.

### Other 2

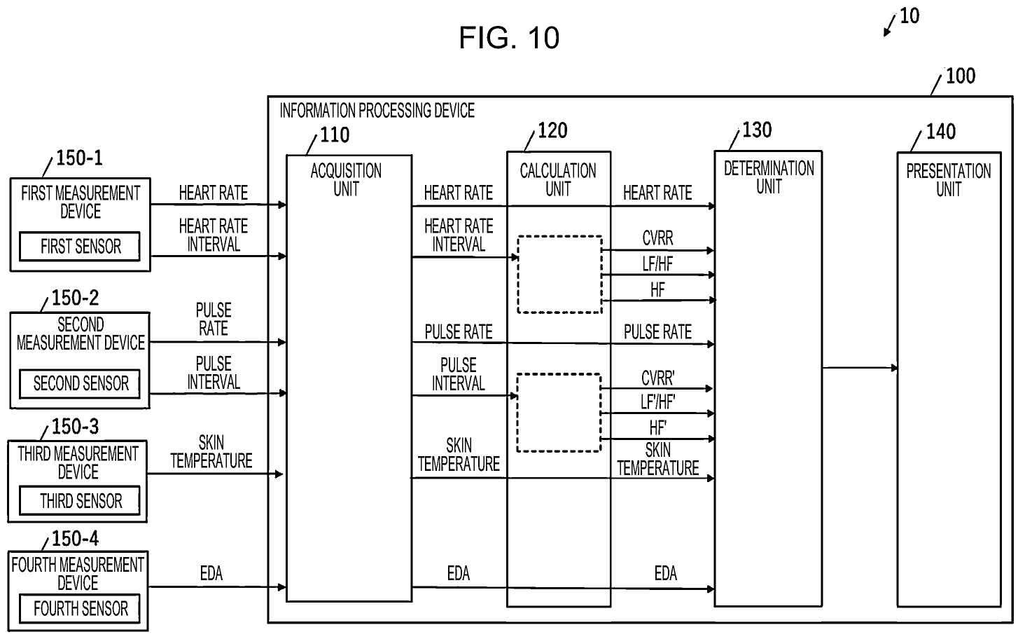

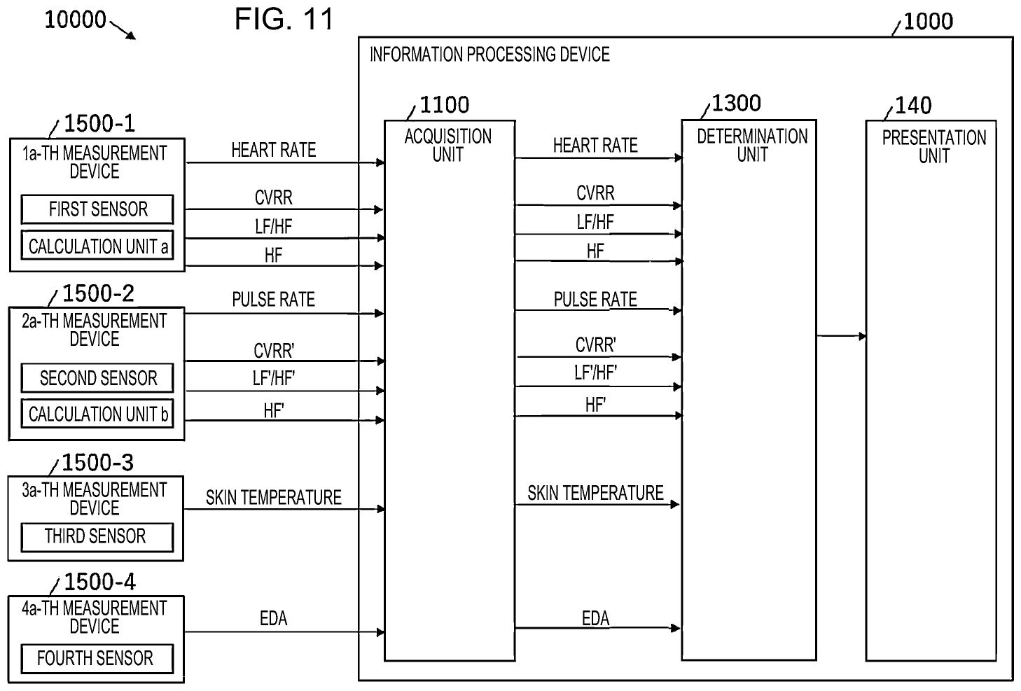

The information processing device 100 may be an information processing device 100 illustrated in FIG. 10 or an information processing device 1000 illustrated in FIG. 11.

### Other 2-1

FIG. 10 is a diagram illustrating an example of transfer of pieces of biometric information in the information processing device 100 according to the embodiment.

The information processing system 10 includes the information processing device 100. The information processing system 10 may include a first measurement device 150-1, a second measurement device 150-2, a third measurement device 150-3, and a fourth measurement device 150-4.

The first measurement device 150-1 includes a first sensor. The first sensor may be an image sensor or one or more electrodes.

The second measurement device 150-2 includes a second sensor. The second sensor may be an image sensor or one or more electrodes.

The third measurement device 150-3 includes a third sensor. The third sensor may be an image sensor or a thermocouple.

The fourth measurement device 150-4 includes a fourth sensor. The fourth sensor may be an EDA sensor. The EDA sensor may include one or more electrodes.

The first measurement device 150-1 may determine a heart rate on the basis of information observed by the first sensor.

The first measurement device 150-1 may generate an electrocardiogram waveform on the basis of information observed by the first sensor. The first measurement device 150-1 may determine heart rate intervals on the basis of the electrocardiogram waveform.

The second measurement device 150-2 may determine a pulse rate on the basis of information observed by the second sensor.

The second measurement device 150-2 may generate an acceleration plethysmogram on the basis of information observed by the second sensor. The second measurement device 150-2 may determine pulse intervals on the basis of the waveform of the acceleration plethysmogram.

The third measurement device 150-3 may determine a skin temperature on the basis of information observed by the third sensor.

The fourth measurement device 150-4 may determine an electrodermal activity on the basis of information observed by the fourth sensor.

The acquisition unit 110 may receive the heart rate and heart rate intervals from the first measurement device 150-1.

The acquisition unit 110 may receive the pulse rate and pulse intervals from the second measurement device 150-2.

The acquisition unit 110 may receive the skin temperature from the third measurement device 150-3.

The acquisition unit 110 may receive the electrodermal activity from the fourth measurement device 150-4.

The acquisition unit 110 may send the heart rate, the heart rate intervals, the pulse rate, the pulse intervals, the skin temperature, and the electrodermal activity to the calculation unit 120. The acquisition unit 110 may send the heart rate, the pulse rate, the skin temperature, and the electrodermal activity to the determination unit 130 without going through the calculation unit 120.

The calculation unit 120 may calculate CVRR, LF/HF, and HF on the basis of the heart rate intervals. The methods for calculating the heart rate intervals, the CVRR, the LF/HF, and the HF are described using FIGS. 1A, 1B, and 1C and Equation 1, for example.

The calculation unit 120 may calculate CVRR′, LF/HF′, and HF′ on the basis of the pulse intervals. A pulse interval is the time interval between two consecutive a-waves in the waveform of the acceleration plethysmogram. Note that the pulse rate is, for example, the number of beats per minute. That is, (the pulse rate)=60/(the pulse interval). Known methods, such as the Pan and Tompkins method, may be used to detect a-waves. The CVRR′ may be equal to (the standard deviation of the pulse intervals)/(the mean of the pulse intervals). The HF′ and the LF may be calculated from the power spectral density obtained by performing frequency analysis using FFT on equally spaced time series data of pulse fluctuations. A power spectrum may be used instead of the power spectral density. The HF′ is an integral value of the power spectral density of a high frequency range (for example, 0.14 Hz to 0.4 Hz). The LF′ is an integral value of the power spectral density of a low frequency range (for example, 0.04 Hz to 0.14 Hz). The frequency conversion using FFT may be performed at 5-second intervals or at any time intervals. The LF′/HF′ may be equal to (the integral value of the above-mentioned power spectral density at 0.04 Hz to 0.14 Hz)/(the integral value of the above-mentioned power spectral density at 0.14 Hz to 0.4 Hz).

Before describing the determination unit 130, a modification of FIG. 3 and that of FIG. 4 will be described.

FIG. 3 may be modified as follows.
In S201, “acquire at least two pieces of biometric information out of the heart rate, pulse rate, LF/HF, HF, skin temperature, and EDA” may be changed to “acquire at least two pieces of biometric information out of the heart rate, pulse rate, LF/HF, HF, LF/HF′, HF′, skin temperature, and EDA”.
In S204, “Do at least two pieces of biometric information out of the heart rate, pulse rate, LF/HF, HF, skin temperature, and EDA indicate that the user is more relaxed in the task execution state than in the resting state?” may be changed to “Do at least two pieces of biometric information out of the heart rate, pulse rate, LF/HF, HF, LF/HF′, HF′, skin temperature, and EDA indicate that the user is more relaxed in the task execution state than in the resting state?”.

FIG. 4 may be modified as follows.
In S301, “acquire CVRR” may be changed to “acquire CVRR, or acquire CVRR”.
In S302, “Is CVRR greater than or equal to the threshold?” may be changed to “Is CVRR greater than or equal to the threshold corresponding to CVRR, or is CVRR′ greater than or equal to a threshold corresponding to CVRR′?”.
In S305, “acquire LF/HF” may be changed to “acquire LF/HF, or acquire LF/HF”.
In S306, “Is LF/HF greater than or equal to the threshold?” may be changed to “Is LF/HF greater than or equal to the threshold corresponding to LF/HF, or is LF/HF′ greater than or equal to a threshold corresponding to LF′/HF′?”.

This concludes the description of the modifications of FIGS. 3 and 4.

The determination unit 130 may receive the heart rate, pulse rate, skin temperature, and electrodermal activity from the acquisition unit 110 via the calculation unit 120. The determination unit 130 may receive the heart rate, pulse rate, skin temperature, and electrodermal activity from the acquisition unit 110 without going through the calculation unit 120. The determination unit 130 may receive the CVRR, LF/HF, HF, CVRR′, LF′/HF′, and HF′ from the calculation unit 120.

The determination unit 130 may receive pieces of biometric information necessary to perform the processes in S204, S302, and S306. That is, the determination unit 130 does not always have to receive all ten pieces of biometric information: heart rate, CVRR, LF/HF, HF, pulse rate, CVRR′, LF′/HF′, HF′, skin temperature, and electrodermal activity. The necessary pieces of biometric information are first biometric information, second biometric information, and third generation information.

The user may instruct the information processing device 100 as to what each of

first biometric information, second biometric information, and third generation information is, and the information processing device 100 may perform S201, S204, S301, S302, S305, and S306 on the basis of the specified first biometric information, second biometric information, and third generation information.

The first biometric information is CVRR or CVRR′.

The second biometric information is LF/HF or LF/HF′.

The third biometric information is one or more pieces of biometric information included in eight pieces of biometric information: heart rate, pulse rate, LF/HF, LF/HF′, HF, HF′, skin temperature, and electrodermal activity.

However, (the second biometric information, the third biometric information) is not equal to (LF/HF, LF/HF), (the second biometric information, the third biometric information) is not equal to (LF/HF′, LF/HF′), (the second biometric information, the third biometric information) is not equal to is not equal to (LF/HF, LF′/HF′), and (the second biometric information, the third biometric information) is not equal to (LF/HF, LF/HF).

Note that the third biometric information does not include the HF or the HF′.

The information processing system 10 does not always have to include all of the first measurement device 150-1, the second measurement device 150-2, the third measurement device 150-3, and the fourth measurement device 150-4. The information processing system 10 may include, among these four measurement devices, one or more measurement devices necessary for the first biometric information, the second biometric information, and the third biometric information to be supplied to the determination unit 130.

The acquisition unit 110 does not always have to acquire all of the heart rate, the heart rate interval, the pulse rate, the pulse interval, the skin temperature, and the electrodermal activity. The acquisition unit 110 may acquire, among these six pieces of biometric information, one or more pieces of biometric information necessary for the first biometric information, the second biometric information, and the third biometric information to be supplied to the determination unit 130.

The acquisition unit 110 does not always have to send all of the heart rate, the heart rate interval, the pulse rate, the pulse interval, the skin temperature, and the electrodermal activity to the calculation unit. Among these six pieces of biometric information, the acquisition unit 110 may send, to the calculation unit 120, one or more pieces of biometric information necessary for the first biometric information, the second biometric information, and the third biometric information to be supplied to the determination unit 130.

The calculation unit 120 does not always have to receive all of the heart rate, the heart rate interval, the pulse rate, the pulse interval, the skin temperature, and the electrodermal activity. The calculation unit 120 may receive, among these six pieces of biometric information, one or more pieces of biometric information necessary for the first biometric information, the second biometric information, and the third biometric information to be supplied to the determination unit 130.

The calculation unit 120 does not always have to calculate all of the six pieces of biometric information, which are CVRR, LF/HF, HF, CVRR′, LF′/HF′, and HF′. Among these six pieces of biometric information, one or more pieces of biometric information that are not included in the first biometric information, the second biometric information, or the third biometric information do not have to be calculated.

The calculation unit 120 does not always have to send all of the heart rate, the CVRR, the LF/HF, the HF, the pulse rate, the CVRR′, the LF′/HF′, the HF′, the skin temperature, and the electrodermal activity to the determination unit. Among these ten pieces of biometric information, the calculation unit 120 may send, to the determination unit 130, pieces of biometric information necessary for the first biometric information, the second biometric information, and the third biometric information to be supplied to the determination unit 130.

The determination unit 130 does not always have to receive all of the heart rate, the CVRR, the LF/HF, the HF, the pulse rate, the CVRR′, the LF/HF′, the HF′, the skin temperature, and the electrodermal activity. The determination unit 130 may receive, among these ten pieces of biometric information, pieces of biometric information identified as the first biometric information, the second biometric information, and the third biometric information.

### Other 2-2

FIG. 11 is a diagram illustrating the information processing device 1000, which is a modification of the information processing device 100 according to the embodiment.

While referring mainly to the differences between the information processing system 10 illustrated in FIG. 10 and including the information processing device 100 described in Other 2-1 and an information processing system 10000 including the information processing device 1000, the information processing device 1000 will be described below.

The information processing system 10000 includes the information processing device 1000. The information processing system 10000 may include a 1a-th measurement device 1500-1, a 2a-th measurement device 1500-2, a 3a-th measurement device 1500-3, and a 4a-th measurement device 1500-4.

The 1a-th measurement device 1500-1 includes a first sensor and a calculation unit a. The first sensor may be an image sensor or one or more electrodes.

The 2a-th measurement device 1500-2 includes a second sensor and a calculation unit b. The second sensor may be an image sensor or one or more electrodes.

The 3a-th measurement device 1500-3 and the third measurement device 150-3 are identical except for a “difference No. 1”. The “difference No. 1” is that the 3a-th measurement device 1500-3 is connected to an acquisition unit 1100, and the third measurement device 150-3 is connected to the acquisition unit 110.

The 4a-th measurement device 1500-4 and the fourth measurement device 150-4 are identical except for a “difference No. 2”. The “difference No. 2” is that the 4a-th measurement device 1500-4 is connected to the acquisition unit 1100, and the fourth measurement device 150-4 is connected to the acquisition unit 110.

The 1a-th measurement device 1500-1 may determine a heart rate on the basis of information observed by the first sensor.

The 1a-th measurement device 1500-1 may generate an electrocardiogram waveform on the basis of the information observed by the first sensor. The 1a-th measurement device 1500-1 may determine heart rate intervals on the basis of the electrocardiogram waveform. The calculation unit a may calculate CVRR, LF/HF, and HF on the basis of the heart rate intervals. These calculation methods are the same as those performed by the calculation unit 120.

The 2a-th measurement device 1500-2 may determine a pulse rate on the basis of information observed by the second sensor.

The 2a-th measurement device 1500-2 may generate an acceleration plethysmogram on the basis of the information observed by the second sensor. The 2a-th measurement device 1500-2 may determine pulse intervals on the basis of the waveform of the acceleration plethysmogram. The calculation unit b may calculate CVRR′, LF/HF′, and HF′ on the basis of the pulse intervals. These calculation methods are the same as those performed by the calculation unit 120.

The acquisition unit 1100 may receive the heart rate, CVRR, LF/HF, and HF from the 1a-th measurement device 1500-1.

The acquisition unit 1100 may receive the pulse rate, CVRR′, LF′/HF′, and HF′ from the 2a-th measurement device 1500-2.

The acquisition unit 1100 may receive the skin temperature from the 3a-th measurement device 1500-3.

The acquisition unit 1100 may receive the electrodermal activity from the 4a-th measurement device 1500-4.

The acquisition unit 1100 may send the heart rate, the CVRR, the LF/HF, the HF, the pulse rate, the CVRR′, the LF′/HF′, the HF′, the skin temperature, and the electrodermal activity to a determination unit 1300.

The modification of FIG. 3 and that of FIG. 4 described in Other 2-1 may be applied to the information processing device 1000.

The determination unit 1300 may receive the heart rate, CVRR, LF/HF, HF, pulse rate, CVRR′, LF/HF′, HF′, skin temperature, and electrodermal activity from the acquisition unit 1100.

The determination unit 130 and the determination unit 1300 are identical except for a “difference No. 3”. The “difference No. 3” is that the determination unit 130 receives the heart rate, CVRR, LF/HF, HF, pulse rate, CVRR′, LF′/HF′, HF, skin temperature, and electrodermal activity from the calculation unit 120, and the determination unit 1300 receives the heart rate, CVRR, LF/HF, HF, pulse rate, CVRR′, LF/HF′, HF′, skin temperature, and electrodermal activity from the acquisition unit 1100.

Matters described below and similar to those referred to in Other 2-1 are applied to the information processing device 1000.
The determination unit 1300 may receive pieces of biometric information necessary to perform the processes in S204, S302, and S306. That is, the determination unit 1300 does not always have to receive all ten pieces of biometric information: heart rate, CVRR, LF/HF, HF, pulse rate, CVRR′, LF/HF′, HF′, skin temperature, and electrodermal activity.
The user may instruct the information processing device 1000 as to what each of first biometric information, second biometric information, and third generation information is, and the information processing device 1000 may perform S201, S204, S301, S302, S305, and S306 on the basis of the specified first biometric information, second biometric information, and third generation information.
The information processing system 10000 does not always have to include all of the 1a-th measurement device 1500-1, the 2a-th measurement device 1500-2, the 3a-th measurement device 1500-3, and the 4a-th measurement device 1500-4. The information processing system 10000 may include, among these four measurement devices, one or more measurement devices necessary for the first biometric information, the second biometric information, and the third biometric information to be supplied to the determination unit 1300.
The acquisition unit 1100 does not always have to acquire all of the heart rate, the CVRR, the LF/HF, the HF, the pulse rate, the CVRR′, the LF′/HF′, the HF′, the skin temperature, and the electrodermal activity. The acquisition unit 1100 may acquire, among these ten pieces of biometric information, pieces of biometric information necessary for the first biometric information, the second biometric information, and the third biometric information to be supplied to the determination unit 1300.
The acquisition unit 1100 done not always have to send all of the heart rate, the CVRR, the LF/HF, the HF, the pulse rate, the CVRR′, the LF′/HF, the HF′, the skin temperature, and the electrodermal activity to the determination unit 1300. Among these ten pieces of biometric information, the acquisition unit 1100 may send pieces of biometric information identified by the first biometric information, second biometric information, and third biometric information to be supplied to the determination unit 1300.
The determination unit 1300 does not always have to receive all of the heart rate, the CVRR, the LF/HF, the HF, the pulse rate, the CVRR′, the LF′/HF′, the HF′, the skin temperature, and the electrodermal activity. It is sufficient that the determination unit 1300 receive, among these ten pieces of biometric information, pieces of biometric information identified by the first biometric information, second biometric information, and third biometric information.

The present disclosure is applicable to devices that sense a person's state.

What is claimed is:

1. An information processing method comprising:
receiving or calculating a coefficient of variation of R-R interval (CVRR) of a user and low frequency/high frequency (LF/HF) of the user; and
causing a processor to perform a determination process for determining, based on the CVRR and the LF/HF, which of a convergent thinking state, a divergent thinking state, or a relaxed state corresponds to a state related to the user's thinking.

2. The information processing method according to claim 1, wherein
in the determination process,
in a case where the CVRR is less than a first threshold, it is determined that the user is in the convergent thinking state.

3. The information processing method according to claim 1, wherein
in the determination process,
in a case where the CVRR is greater than or equal to a first threshold and where the LF/HF is greater than or equal to a second threshold, it is determined that the user is in the divergent thinking state.

4. The information processing method according to claim 1, wherein
in the determination process,
in a case where the CVRR is greater than or equal to a first threshold and where the LF/HF is less than a second threshold, it is determined that the user is in the relaxed state.

5. The information processing method according to claim 1, further comprising:
before the determination process, causing the processor to perform a pre-determination process for determining, based on at least the LF/HF, whether or not a state of the user is one of the convergent thinking state, the divergent thinking state, or the relaxed state, wherein
in a case where the state of the user is determined to be one of the convergent thinking state, the divergent thinking state, or the relaxed state in the pre-determination process, the determination process is performed.

6. The information processing method according to claim 1, further comprising:
before the determination process, causing the processor to perform a pre-determination process for determining, based on at least one of a heart rate, a pulse rate, the LF/HF, HF, a skin temperature, or an electrodermal activity of the user, whether or not a state of the user is one of the convergent thinking state, the divergent thinking state, or the relaxed state, wherein
in a case where the state of the user is determined to be one of the convergent thinking state, the divergent thinking state, or the relaxed state in the pre-determination process, the determination process is performed.

7. The information processing method according to claim 1, wherein
the CVRR and the LF/HF correspond to a first period included in periods, and in the determination process,
based on the CVRR and LF/HF corresponding to each of the periods, the determination process regarding the state related to the user's thinking in the period is performed,
the information processing method, further comprising:
generating a graph representing a percentage of a period determined to be in the convergent thinking state, a percentage of a period determined to be in the divergent thinking state, and a percentage of a period determined to be in the relaxed state among the periods in the determination process; and
displaying the graph.

8. The information processing method according to claim 2, further comprising:
acquiring identification information for identifying the user; and
acquiring threshold information corresponding to the identification information, wherein
the first threshold is determined based on the threshold information.

9. The information processing method according to claim 8, further comprising:
acquiring evaluation information obtained by evaluating the state related to the user's thinking using a method different from the determination process; and
updating the threshold information based on the determined state of the user and a state of the user indicated by the evaluation information.

10. The information processing method according to claim 3, further comprising:
acquiring identification information for identifying the user; and
acquiring threshold information corresponding to the identification information, wherein
the first threshold and the second threshold are determined based on the threshold information.

11. The information processing method according to claim 10, further comprising:
acquiring evaluation information obtained by the user evaluating the state related to the user's thinking; and
updating the threshold information based on the determined state of the user and the state of the user indicated by the evaluation information.

12. The information processing method corresponding to claim 2, further comprising:
after performing the determination process, performing
(i) control to lower a color temperature of illuminating light and increase illuminance of the illuminating light, or
(ii) control to lower a color temperature of illuminating light and lower illuminance of the illuminating light
on a lighting device illuminating a surrounding area of the user.

13. The information processing method according to claim 3, further comprising:
after performing the determination process, performing
(i) control to increase a color temperature of illuminating light and increase illuminance of the illuminating light, or
(ii) control to lower a color temperature of illuminating light and lower illuminance of the illuminating light
on a lighting device illuminating a surrounding area of the user.

14. The information processing method according to claim 4, further comprising:
after performing the determination process, performing
(i) control to lower a color temperature of illuminating light and increase illuminance of the illuminating light, or
(ii) control to increase a color temperature of illuminating light and increase illuminance of the illuminating light
on a lighting device illuminating a surrounding area of the user.

15. An information processing method comprising:
by using a processor,
acquiring target information indicating a target state that is a state targeted by a user;
acquiring biometric information of the user;
determining a state related to the user's thinking based on the biometric information;
comparing the target state indicated by the target information with the determined state related to the user's thinking; and
in a case where, as a result of the comparison, the target state and the determined state related to the user's thinking are different from each other, performing control to change characteristics of illuminating light emitted from a lighting device illuminating a surrounding area of the user.

16. An information processing system comprising:
a light source that irradiates a user with irradiation light;
a detector that detects reflected light generated due to the irradiation light;
a processing circuit;
a memory device; and
a display device, wherein
the processing circuit
calculates a CVRR corresponding to heart rate fluctuations or pulse fluctuations of the user by performing a time-domain analysis process on a component of the reflected light detected by the detector over a unit period of time,
calculates an LF/HF corresponding to the heart rate fluctuations or pulse fluctuations of the user by performing a frequency analysis process on the component of the reflected light detected by the detector over the unit period of time,
acquires threshold information indicating a first threshold and a second threshold from the memory device,
performs a first determination process for determining whether or not a mean value of the CVRR in the unit period of time is greater than or equal to the first threshold,
performs a second determination process for determining whether or not a mean value of the LF/HF in the unit period of time is greater than or equal to the second threshold,
performs a third determination process for determining, based on the first determination process and the second determination process, which of a divergent thinking state, a convergent thinking state, or a relaxed state the user is in during the unit period of time,
generates an image related to the state of the user determined in the third determination process, and causes the display device to display the generated image.

17. The information processing method according to claim 1, wherein
the CVRR is calculated based on a first standard deviation of heart rate intervals of the user and a first mean of the heart rate intervals, and
the LF/HF is calculated based on a first integral value of a first section of a power spectral density of the heart rate intervals of the user based on the heart rate intervals and a second integral value of a second section of the power spectral density.

18. The information processing method according to claim 17, wherein
the CVRR is (the first standard deviation)/(the first mean),
the LF/HF is (the first integral value)/(the second integral value),
the sensor has electrodes for measuring an electrocardiogram waveform of the user,
the heart rate intervals are determined based on the electrocardiogram waveform,
each of the heart rate intervals is a time interval between adjacent R waves in the electrocardiogram waveform,
a beginning of the first section is a first frequency, an end of the first section is a second frequency,
a beginning of the second section is a third frequency, an end of the second section is a fourth frequency,
the first frequency is lower than the second frequency, the third frequency is lower than the fourth frequency,
the first frequency is lower than the third frequency, and the second frequency is lower than the fourth frequency.

19. The information processing method according to claim 16, wherein
the CVRR is calculated based on a first standard deviation of intervals and a first mean of the intervals,
the LF/HF is calculated based on a first integral value of a first section of a power spectral density and a second integral value of a second section of the power spectral density,
the intervals are heart rate intervals of the user or pulse intervals of the user,
the power spectral density is a first power spectral density of the heart rate intervals of the user based on the heart rate intervals or a second power spectral density of the pulse intervals of the user based on the pulse intervals.

20. The information processing method according to claim 19, wherein
the CVRR is (the first standard deviation)/(the first mean),
the LF/HF is (the first integral value)/(the second integral value),
a beginning of the first section is a first frequency, an end of the first section is a second frequency,
a beginning of the second section is a third frequency, an end of the second section is a fourth frequency,
the first frequency is lower than the second frequency. the third frequency is lower than the fourth frequency.
the first frequency is lower than the third frequency, and the second frequency is lower than the fourth frequency.
